(19) United States
(12) Patent Application Publication
(10) Pub. No.: US 2011/0098322 A1
(43) Pub. Date: Apr. 28, 2011

(54) PREPARATION OF NOVEL 1,3-SUBSTITUTED UREAS AS INHIBITORS OF SOLUBLE EPOXIDE HYDROLASE

(75) Inventors: James R. Sanborn, Sacramento, CA (US); Bruce D. Hammock, Davis, CA (US); Sung Hee Hwang, Davis, CA (US); Paul D. Jones, Aberdeen, NJ (US); Christophe Morisseau, West Sacramento, CA (US)

(73) Assignee: THE REGENTS OF THE UNIVERSITY OF CALIFORNIA, Oakland, CA (US)

(21) Appl. No.: 12/672,405

(22) PCT Filed: Aug. 5, 2008
(86) PCT No.: PCT/US08/72199

§ 371 Date: Jan. 6, 2011

Related U.S. Application Data
(60) Provisional application No. 60/954,157, filed on Aug. 6, 2007.

(51) Int. Cl. A61K 31/4468; C07C 275/26; C07C 255/60; C07C 275/24; C07C 229/66; C07D 211/32; C07D 211/58; C07D 211/96; C12N 9/99; A61K 31/17; A61K 31/277; A61K 31/196; A61K 31/24; A61K 31/445

(52) U.S. Cl. 514/329; 564/56; 558/417; 562/439; 560/34; 546/226; 546/224; 435/184; 514/595; 514/521; 514/563; 514/535; 514/330

(57) ABSTRACT

The present invention provides compounds that can inhibit the activity of soluble epoxide hydrolases. In particular, the present invention provides compounds of Formula I.

### CROSS-REFERENCES TO RELATED APPLICATIONS

This application claims priority to U.S. Provisional Application No. 60/954,157, filed Aug. 6, 2007, incorporated herein in its entirety.

### STATEMENT AS TO RIGHTS TO INVENTIONS MADE UNDER FEDERALLY SPONSORED RESEARCH AND DEVELOPMENT

This invention was made with Government support under Grant Nos. R37 ES02710 and P42 ES004699, awarded by the National Institutes of Environmental Health Sciences. The Government has certain rights in this invention.

### BACKGROUND OF THE INVENTION

Epoxide hydrolases (EHs, EC 3.3.2.3) catalyze the hydrolysis of epoxides or arene oxides to their corresponding diols by the addition of water (see, Oesch, F., et al., Xenobiotica 1973, 3, 305-340). EHs play an important role in the metabolism of a variety of compounds including hormones, chemotherapeutic drugs, carcinogens, environmental pollutants, mycotoxins, and other harmful foreign compounds.

There are two well-studied EHs, microsomal epoxide hydrolase (mEH) and soluble epoxide hydrolase (sEH). These enzymes are very distantly related, have different subcellular localization, and have different but partially overlapping substrate selectivities. The soluble and microsomal EH forms are known to complement each other in detoxifying a wide array of mutagenic, toxic, and carcinogenic xenobiotic epoxides (see, Hammock, B. D., et al., COMPREHENSIVE TOXICOLOGY. Oxford: Pergamon Press 1997, 283-305; Fretland, A. J., et al., Chem. Biol. Intereract 2000, 129, 41-59 and Morisseau, C. and B. D. Hammock. 2008. Pest. Manag. Sci. 64:594-609).

The sEH is also involved in the metabolism of arachidonic acid (see, Zeldin, D. C., et al., J. Biol. Chem. 1993, 268, 6402-6407), linoleic (see, Moghaddam, M. F., et al., Nat. Med. 1997, 3, 562-567) acid, and other lipid epoxides, some of which are endogenous chemical mediators (see, Carroll, M. A., et al., Thorax 2000, 55, S13-16; Newman, J. W., C. Morisseau and B. D. Hammock, Prog. Lipid Res. 2005, 44:1-51). Epoxides of arachidonic acid (epoxyeicosatrienoic acids or EETs) are known effectors of blood pressure (see, Capdevila, J. H., et al., J. Lipid. Res. 2000, 41, 163-181), and modulators of vascular permeability (see, Oltman, C. L., et al., Circ Res. 1998, 83, 932-939). The vasodilatory properties of EETs are associated with an increased open-state probability of calcium-activated potassium channels leading to hyperpolarization of the vascular smooth muscle (see Fisslthaler, B., et al., Nature 1999, 401, 493-497). Hydrolysis of the epoxides by sEH diminishes this activity (see, Capdevila, J. H., et al., J. Lipid. Res. 2000, 41, 163-181). sEH hydrolysis of EETs also regulates their incorporation into coronary endothelial phospholipids, suggesting a regulation of endothelial function by sEH (see, Weintraub, N. L., et al., Am. J. Physiol. 1992, 277, H2098-2108). It has recently been shown that treatment of spontaneous hypertensive rats (SHRs) with selective sEH inhibitors significantly reduces their blood pressure (see, Yu, Z., et al., Circ. Res. 2000, 87, 992-998). In addition, male knockout sEH mice have significantly lower blood pressure than wild-type mice (see Sinal, C. J., et al., J. Biol. Chem. 2000, 275, 40504-405010; Chiamvimonvat, N., C.-M. Ho, H.-J. Tsai and B. D. Hammock, J. Carviovasc. Pharm. 2007, 50:225-237), further supporting the role of sEH in blood pressure regulation.

The EETs have also demonstrated anti-inflammatory properties in endothelial cells (see, Node, K., et al., Science 1999, 285, 1276-1279 and Campbell, W. B. Trends Pharmacol. Sci. 2000, 21, 125-127). In contrast, diols derived from epoxy-linoleate (leukotoxin) perturb membrane permeability and calcium homeostasis (see, Moghaddam, M. F., et al., Nat. Med. 1997, 3, 562-567), which results in inflammation that is modulated by nitric oxide synthase and endothelin-1 (see, Ishizaki, T., et al., Am. J. Physiol. 1995, 269, L65-70 and Ishizaki, T., et al., J. Appl. Physiol. 1995, 79, 1106-1611). Micromolar concentrations of leukotoxin reported in association with inflammation and hypoxia (see, Dudda, A., et al., Chem. Phys. Lipids 1996, 82, 39-51), depress mitochondrial respiration in vitro (see, Sakai, T., et al., Am. J. Physiol. 1995, 269, L326-331), and cause mammalian cardiopulmonary toxicity in vivo (see, Ishizaki, T., et al., Am. J. Physiol. 1995, 269, L65-70; Fukushima, A., et al., Cardiovasc. Res. 1988, 22, 213-218; and Ishizaki, T., et al., Am. J. Physiol. 1995, 268, L123-128).

Leukotoxin toxicity presents symptoms suggestive of multiple organ failure and acute respiratory distress syndrome (ARDS) (see, Ozawa, T. et al., Am. Rev. Respir. Dis. 1988, 137, 535-540). In both cellular and organismal models, leukotoxin-mediated toxicity is dependent upon epoxide hydrolysis (see, Moghaddam, M. F., et al., Nat. Med. 1997, 3, 562-567; Morisseau, C., et al., Proc. Natl. Acad. Sci. USA 1999, 96, 8849-8854; and Zheng, J., et al., Am. J. Respir. Cell Mol. Biol. 2001, 25, 434-438), suggesting a role for sEH in the regulation of inflammation. The bioactivity of these epoxy-fatty acids suggests that inhibition of vicinal-dihydroxy-lipid biosynthesis may have therapeutic value, making sEH a promising pharmacological target (Schmelzer, K. R., B. Inceoglu, L. Kubala, I.-H. Kim, S. L. Jinks, J. P. Eiserich and B. D. Hammock, Proc. Natl. Acad. Sci. USA. 2006, 103:13646-13651; B., S. L. Jinks, K. R. Schmelzer, T. Waite, I.-H. Kim and B. D. Hammock, Life Sci. 2006 79:2311-2319).

Recently, 1,3-disubstituted ureas, carbamates, and amides have been reported as new potent and stable inhibitors of sEH. See, U.S. Pat. No. 6,150,415. These compounds are competitive tight-binding inhibitors with nanomolar K_{I} values that interact stoichiometrically with purified recombinant sEH (see, Morisseau, C., et al., Proc. Natl. Acad. Sci. USA 1999, 96, 8849-8854). Based on the X-ray crystal structure, the urea inhibitors were shown to establish hydrogen bonds and to form salt bridges between the urea function of the inhibitor and residues of the sEH active site, mimicking features encountered in the reaction coordinate of epoxide ring opening by this enzyme (see, Argiriadi, M. A., et al., Proc. Natl. Acad. Sci. USA 1999, 96, 10637-10642 and Argiriadi, M. A., et al., J. Biol. Chem. 2000, 275, 15265-15270). These inhibitors efficiently reduced epoxide hydrolysis in several in vitro and in vivo models (see, Yu, Z., et al., Circ. Res. 2000, 87, 992-998; Morisseau, C., et al., Proc. Natl. Acad. Sci. USA 1999, 96, 8849-8854; and Newman, J. W., et al., Environ. Health Perspect. 2001, 109, 61-66). Despite the activity associated with these inhibitors, there exists a need for compounds possessing similar or increased activities, with improved solubility to facilitate formulation and delivery (Hwang, S. H., H. J. Tsai, J.-Y. Liu, C. Morisseau and B. D. Hammock, J. Med. Chem. 2007, 50(16):3825-3840; Kim, I.-H., H.-J. Tsai, K. Nishi, T. Kasagami, C. Morisseau and B. D. Hammock, J. Med. Chem. 2007, 50:5217-5226).

Surprisingly, the present invention provides such compounds along with methods for their use and compositions that contain them.

### BRIEF SUMMARY OF THE INVENTION

In one embodiment, the present invention provides a compound having a formula:

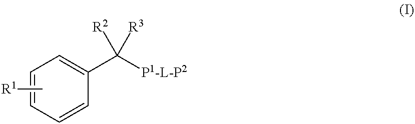

and the pharmaceutically acceptable salts, wherein R¹ is C₁-C₆ alkyl, C₁-C₆ hydroxyalkyl, C₁-C₆ haloalkyl, C₂-C₆ alkenyl, —C(O)—C₁-C₆ alkyl, C₁-C₆ alkyl-OSO₃H, C₃-C₆ cycloalkyl or an epoxy group optionally substituted with 1-2 groups each independently H or C₁₋₆ alkyl. Each of R² and R³ are independently C₁-C₆ alkyl or C₂-C₆ alkenyl, or R² and R³ are optionally combined to form a C₃-C₆ cycloalkyl. P¹ is a primary pharmacophore of the formula —NH—C(O)—NH—. L is C₁-C₁₂ alkylene, C₃-C₆ cycloalkylene, aryl-C₀-C₆ alkylene, C₃-C₆ cycloalkylene-O-aryl, C₀-C₆alkylenearyl-O-aryl or
C₀-C₆-alkylene-C₃-C₆-heterocycloalkylene. P² is C₂-C₆ alkenyl, C₂-C₆ alkynyl, C₁-C₆ haloalkyl, aryl, heteroaryl, heterocyclyl, —O(CH₂CH₂O)_{q}—R⁴, —OR⁴, —CN, —C(O)NHR⁴, —C(O)NHS(O)₂R⁴, —NHS(O)₂R⁴, —O—C₂-C₄alkyl-C(O)OR⁴, —C(O)R⁴, —C(O)OR⁴ or carboxylic acid analogs, wherein R⁴ is hydrogen, C₁-C₆ alkyl, C₃-C₈ cycloalkyl, heterocyclyl, aryl or aryl-C₁-C₄ alkyl, or optionally P² is H. Subscript q is from 1 to 6.

In a second embodiment, the present invention provides a pharmaceutical composition comprising a compound of Formula I and a pharmaceutically acceptable excipient.

In a third embodiment, the present invention provides a method for inhibiting a soluble epoxide hydrolase, comprising contacting said soluble epoxide hydrolase with an inhibiting amount of a compound of Formula I.

### BRIEF DESCRIPTION OF THE DRAWINGS

FIG. 1 shows the in vivo PK profile of 1956.

### DETAILED DESCRIPTION OF THE INVENTION

### I. ABBREVIATIONS AND DEFINITIONS

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO:1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

The terms “treat”, “treating” and “treatment” refer to any method of alleviating or abrogating a disease or its attendant symptoms.

The term “therapeutically effective amount” refers to that amount of the compound being administered sufficient to prevent or decrease the development of one or more of the symptoms of the disease, condition or disorder being treated.

The term “modulate” refers to the ability of a compound to increase or decrease the function, or activity, of the associated activity (e.g., soluble epoxide hydrolase). “Modulation”, as used herein in its various forms, is meant to include antagonism and partial antagonism of the activity associated with sEH. Inhibitors of sEH are compounds that, e.g., bind to, partially or totally block the enzyme's activity.

The term “compound” as used herein is intended to encompass not only the specified molecular entity but also its pharmaceutically acceptable, pharmacologically active derivatives, including, but not limited to, salts, prodrug conjugates such as esters and amides, metabolites, hydrates, solvates and the like.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The “subject” is defined herein to include animals such as mammals, including, but not limited to, primates (e.g., humans), cows, sheep, goats, horses, dogs, cats, rabbits, rats, mice and the like. In some embodiments, the subject is a human.

As used herein, the term “sEH-mediated disease or condition” and the like refers to a disease or condition characterized by less than or greater than normal, sEH activity. A sEH-mediated disease or condition is one in which modulation of sEH results in some effect on the underlying condition or disease (e.g., a sEH inhibitor or antagonist results in some improvement in patient well-being in at least some patients).

“Parenchyma” refers to the tissue characteristic of an organ, as distinguished from associated connective or supporting tissues.

“Chronic Obstructive Pulmonary Disease” or “COPD” is also sometimes known as “chronic obstructive airway disease”, “chronic obstructive lung disease”, and “chronic airways disease.” COPD is generally defined as a disorder characterized by reduced maximal expiratory flow and slow forced emptying of the lungs. COPD is considered to encompass two related conditions, emphysema and chronic bronchitis. COPD can be diagnosed by the general practitioner using art recognized techniques, such as the patient's forced vital capacity (“FVC”), the maximum volume of air that can be forcibly expelled after a maximal inhalation. In the offices of general practitioners, the FVC is typically approximated by a 6 second maximal exhalation through a spirometer. The definition, diagnosis and treatment of COPD, emphysema, and chronic bronchitis are well known in the art and discussed in detail by, for example, Honig and Ingram, in Harrison's Principles of Internal Medicine, (Fauci et al., Eds.), 14th Ed., 1998, McGraw-Hill, New York, pp. 1451-1460 (hereafter, “Harrison's Principles of Internal Medicine”).

“Emphysema” is a disease of the lungs characterized by permanent destructive enlargement of the airspaces distal to the terminal bronchioles without obvious fibrosis.

“Chronic bronchitis” is a disease of the lungs characterized by chronic bronchial secretions which last for most days of a month, for three months a year, for two years.

As the names imply, “obstructive pulmonary disease” and “obstructive lung disease” refer to obstructive diseases, as opposed to restrictive diseases. These diseases particularly include COPD, bronchial asthma and small airway disease.

“Small airway disease.” There is a distinct minority of patients whose airflow obstruction is due, solely or predominantly to involvement of the small airways. These are defined as airways less than 2 mm in diameter and correspond to small cartilaginous bronchi, terminal bronchioles and respiratory bronchioles. Small airway disease (SAD) represents luminal obstruction by inflammatory and fibrotic changes that increase airway resistance. The obstruction may be transient or permanent.

The “interstitial lung diseases (ILDs)” are a group of conditions involving the alveolar walls, perialveolar tissues, and contiguous supporting structures. As discussed on the website of the American Lung Association, the tissue between the air sacs of the lung is the interstitium, and this is the tissue affected by fibrosis in the disease. Persons with the disease have difficulty breathing in because of the stiffness of the lung tissue but, in contrast to persons with obstructive lung disease, have no difficulty breathing out. The definition, diagnosis and treatment of interstitial lung diseases are well known in the art and discussed in detail by, for example, Reynolds, H. Y., in Harrison's Principles of Internal Medicine, supra, at pp. 1460-1466. Reynolds notes that, while ILDs have various initiating events, the immunopathological responses of lung tissue are limited and the ILDs therefore have common features.

“Idiopathic pulmonary fibrosis,” or “IPF,” is considered the prototype ILD. Although it is idiopathic in that the cause is not known, Reynolds, supra, notes that the term refers to a well defined clinical entity.

“Bronchoalveolar lavage,” or “BAL,” is a test which permits removal and examination of cells from the lower respiratory tract and is used in humans as a diagnostic procedure for pulmonary disorders such as IPF. In human patients, it is usually performed during bronchoscopy.

As used herein, the term “alkyl” refers to a saturated hydrocarbon radical which may be straight-chain or branched-chain (for example, ethyl, isopropyl, t-amyl, or 2,5-dimethylhexyl). This definition applies both when the term is used alone and when it is used as part of a compound term, such as “hydroxyalkyl,” “haloalkyl,” “arylalkyl,” “alkylamino” and similar terms. In some embodiments, alkyl groups are those containing 1 to 24 carbon atoms. All numerical ranges in this specification and claims are intended to be inclusive of their upper and lower limits. Additionally, the alkyl and heteroalkyl groups may be attached to other moieties at any position on the alkyl or heteroalkyl radical which would otherwise be occupied by a hydrogen atom (such as, for example, 2-pentyl, 2-methylpent-1-yl and 2-propyloxy). Divalent alkyl groups may be referred to as “alkylene,” and divalent heteroalkyl groups may be referred to as “heteroalkylene,” such as those groups used as linkers in the present invention. The alkyl, alkylene, and heteroalkylene moieties may also be optionally substituted with halogen atoms, or other groups such as oxo, cyano, nitro, alkyl, alkylamino, carboxyl, hydroxyl, alkoxy, aryloxy, and the like.

As used herein, the term “haloalkyl” refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, flouromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

As used herein, the term “epoxy group” refers to a 3-membered heterocyclic ring structure having one oxygen atom. The epoxy group can be substituted with a variety of substituents including, but not limited to, alkyl.

The terms “cycloalkyl” and “cycloalkylene” refer to a saturated hydrocarbon ring and includes bicyclic and polycyclic rings. Similarly, cycloalkyl and cycloalkylene groups having a heteroatom (e.g. N, O or S) in place of a carbon ring atom may be referred to as “heterocycloalkyl” and “heterocycloalkylene,” respectively. Examples of cycloalkyl and heterocycloalkyl groups are, for example, cyclohexyl, norbornyl, adamantyl, morpholinyl, thiomorpholinyl, dioxothiomorpholinyl, and the like. The cycloalkyl and heterocycloalkyl moieties may also be optionally substituted with halogen atoms, or other groups such as nitro, alkyl, alkylamino, carboxyl, alkoxy, aryloxy and the like. In some embodiments, cycloalkyl and cycloalkylene moieties are those having 3 to 12 carbon atoms in the ring (e.g., cyclohexyl, cyclooctyl, norbornyl, adamantyl, and the like). In some embodiments, heterocycloalkyl and heterocycloalkylene moieties are those having 1 to 3 hetero atoms in the ring (e.g., morpholinyl, thiomorpholinyl, dioxothiomorpholinyl, piperidinyl and the like). Additionally, the term “(cycloalkyl)alkyl” refers to a group having a cycloalkyl moiety attached to an alkyl moiety. Examples are cyclohexylmethyl, cyclohexylethyl and cyclopentylpropyl.

The term “alkenyl” as used herein refers to an alkyl group as described above which contains one or more sites of unsaturation that is a double bond. Similarly, the term “alkynyl” as used herein refers to an alkyl group as described above which contains one or more sites of unsaturation that is a triple bond.

The term “alkoxy” refers to an alkyl radical as described above which also bears an oxygen substituent which is capable of covalent attachment to another hydrocarbon radical (such as, for example, methoxy, ethoxy and t-butoxy).

The term “aryl” refers to an aromatic carbocyclic substituent which may be a single ring or multiple rings which are fused together, linked covalently or linked to a common group such as an ethylene or methylene moiety. Similarly, aryl groups having a heteroatom (e.g. N, O or S) in place of a carbon ring atom are referred to as “heteroaryl”. Examples of aryl and heteroaryl groups are, for example, phenyl, naphthyl, biphenyl, diphenylmethyl, thienyl, pyridyl and quinoxalyl. The aryl and heteroaryl moieties may also be optionally substituted with halogen atoms, or other groups such as nitro, alkyl, alkylamino, carboxyl, alkoxy, phenoxy and the like. Additionally, the aryl and heteroaryl groups may be attached to other moieties at any position on the aryl or heteroaryl radical which would otherwise be occupied by a hydrogen atom (such as, for example, 2-pyridyl, 3-pyridyl and 4-pyridyl). Divalent aryl groups are “arylene”, and divalent heteroaryl groups are referred to as “heteroarylene” such as those groups used as linkers in the present invention.

The terms “arylalkyl” and “alkylaryl”, “refer to an aryl radical attached directly to an alkyl group. Likewise, the terms “arylalkenyl” and “aryloxyalkyl” refer to an alkenyl group, or an oxygen which is attached to an alkyl group, respectively. For brevity, aryl as part of a combined term as above, is meant to include heteroaryl as well. The term “aryloxy” refers to an aryl radical as described above which also bears an oxygen substituent which is capable of covalent attachment to another radical (such as, for example, phenoxy, naphthyloxy, and pyridyloxy).

The terms “halo” or “halogen,” by themselves or as part of another substituent, mean, unless otherwise stated, a fluorine, chlorine, bromine, or iodine atom. Additionally, terms such as “haloalkyl,” and “haloalkoxy” are meant to include monohaloalkyl(oxy) and polyhaloalkyl(oxy). For example, the term “C₁-C₆ haloalkyl” is mean to include trifluoromethyl, 2,2,2-trifluoroethyl, 4-chlorobutyl, 3-bromopropyl, and the like.

The term “hetero” as used in a “heteroatom-containing alkyl group” (a “heteroalkyl” group) or a “heteroatom-containing aryl group” (a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur or more than one non-carbon atom (e.g., sulfonamide). Similarly, the term “heteroalkyl” refers to an alkyl substituent that is heteroatom-containing, the terms “heterocyclic” “heterocycle” or “heterocyclyl” refer to a cyclic substituent or group that is heteroatom-containing and is either aromatic or non-aromatic. The terms “heteroaryl” and “heteroaromatic” respectively refer to “aryl” and “aromatic” substituents that are heteroatom-containing, and the like. The terms “heterocyclic” and “heterocyclyl” include the terms “heteroaryl” and “heteroaromatic”. In some embodiments, heterocyclic moieties are those having 1 to 3 hetero atoms in the ring. Examples of heteroalkyl groups include alkoxy, alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing cyclic nonaromatic groups are morpholinyl, piperazinyl, piperidinyl, etc.

The term “carboxylic acid analog” refers to a variety of groups having an acidic moiety that are capable of mimicking a carboxylic acid residue. Examples of such groups are sulfonic acids, sulfinic acids, phosphoric acids, phosphonic acids, phosphinic acids, sulfonamides, and heterocyclic moieties such as, for example, imidazoles, triazoles and tetrazoles.

The term “salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The term “substituted” refers to the replacement of an atom or a group of atoms of a compound with another atom or group of atoms. For example, an atom or a group of atoms may be substituted with one or more of the following substituents or groups: halo, nitro, C₁-C₈alkyl, C₁-C₈alkylamino, hydroxyC₁-C₈alkyl, haloC₁-C₈alkyl, carboxyl, hydroxyl, C₁-C₈alkoxy, C₁-C₈alkoxyC₁-C₈alkoxy, thioC₁-C₈alkyl, aryl, aryloxy, C₃-C₈cycloalkyl, C₃-C₈cycloalkyl C₁-C₈alkyl, heteroaryl, arylC₁-C₈alkyl, heteroarylC₁-C₈alkyl, C₂-C₈alkenyl containing 1 to 2 double bonds, C₂-C₈alkynyl containing 1 to 2 triple bonds, C₄-C₈alk(en)(yn)yl groups, cyano, formyl, C₁-C₈alkylcarbonyl, arylcarbonyl, heteroarylcarbonyl, C₁-C₈alkoxycarbonyl, aryloxycarbonyl, aminocarbonyl, C₁-C₈alkylaminocarbonyl, C₁-C₈dialkylaminocarbonyl, aryl aminocarbonyl, diarylaminocarbonyl, arylC₁-C₈alkylaminocarbonyl, haloC₁-C₈alkoxy, C₂-C₈alkenyloxy, C₂-C₈alkynyloxy, arylC₁-C₈alkoxy, aminoC₁-C₈alkyl, C₁-C₈alkylaminoC₁-C₈alkyl, C₁-C₈dialkylaminoC₁-C₈alkyl, arylaminoC₁-C₈alkyl, amino, C₁-C₈dialkylamino, arylamino, arylC₁-C₈alkylamino, C₁-C₈alkylcarbonylamino, arylcarbonylamino, azido, mercapto, C₁-C₈alkylthio, arylthio, haloC₁-C₈alkylthio, thiocyano, isothiocyano, C₁-C₈alkylsulfinyl, C₁-C₈alkylsulfonyl, arylsulfinyl, arylsulfonyl, aminosulfonyl, C₁-C₈alkylaminosulfonyl, C₁-C₈dialkylaminosulfonyl and arylaminosulfonyl. When the term “substituted” appears prior to a list of possible substituted groups, it is intended that the term apply to every member of that group.

The term “unsubstituted” refers to a native compound that lacks replacement of an atom or a group of atoms.

The term “contacting” refers to the process of bringing into contact at least two distinct species such that they can react. It should be appreciated, however, the resulting reaction product can be produced directly from a reaction between the added reagents or from an intermediate from one or more of the added reagents which can be produced in the reaction mixture.

### II. GENERAL

The present invention derives from the discovery that 1,3-disubstituted ureas (or the corresponding amides or carbamates, also referred to as the primary pharmacophore) can be further functionalized to provide more potent sEH inhibitors with improved physical properties. As described herein, benzylic carbons and carbons alpha to heteroatoms, such as the urea nitrogen, can be rapidly oxidized. The gem-dimethyl groups of some compounds of the present invention illustrate the concept that by blocking the unstable site, the pharmacokinetic properties can be improved.

The discovery of the gem-dimethyl pharmacophores has also led to the employment of combinatorial chemistry approaches for establishing a wide spectrum of compounds having sEH inhibitory activity. The polar pharmacophores divide the molecule into domains each of which can be easily manipulated by common chemical approaches in a combinatorial manner, leading to the design and confirmation of novel orally available therapeutic agents for the treatment of diseases such as hypertension and vascular inflammation. The agents of the present invention treat such diseases,while simultaneously increasing sodium excretion, reducing vascular and renal inflammation, and reducing male erectile dysfunction. Alterations in solubility, bioavailability and pharmacological properties leads to compounds that can alter the regulatory lipids of experimental animals increasing the relative amounts of epoxy arachidonate derivatives when compared either to their diol products or to the proinflammatory and hypertensive hydroxyeicosatetraenoic acids (HETEs). Since epoxy arachidonates are anti-hypertensive and anti-inflammatory, among other beneficial properties, altering the lipid ratios can lead to reduced blood pressure and reduced vascular and renal inflammation. This approach has been validated as reported in U.S. patent application Ser. Nos. 10/817,334 and 11/256,685 which are herein incorporated by reference in their entirety.

### III. COMPOUNDS FOR INHIBITING SOLUBLE EPOXIDE HYDROLASES

In some embodiments, the present invention provides compounds that can inhibit the activity of soluble epoxide hydrolases. In particular, the present invention provides compounds of Formula I:

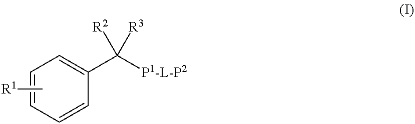

and the pharmaceutically acceptable salts, wherein R¹ is C₁-C₆ alkyl, C₁-C₆ hydroxyalkyl, C₁-C₆ haloalkyl, C₂-C₆ alkenyl, —C(O)—C₁-C₆ alkyl, C₁-C₆ alkyl-OSO₃H, C₃-C₆ cycloalkyl or an epoxy group optionally substituted with 1-2 groups each independently H or C₁₋₆ alkyl. Each of R² and R³ are independently C₁-C₆ alkyl or C₂-C₆ alkenyl, or R² and R³ are optionally combined to form a C₃-C₆ cycloalkyl. P¹ is a primary pharmacophore of the formula —NH—C(O)—NH—. L is C₁-C₁₂ alkylene, C₃-C₆ cycloalkylene, aryl-C₀-C₆ alkylene, C₃-C₆ cycloalkylene-O-aryl, C₀-C₆alkylenearyl-O-aryl or
C₀-C₆-alkylene-C₃-C₆-heterocycloalkylene. P² is C₂-C₆ alkenyl, C₂-C₆ alkynyl, C₁-C₆ haloalkyl, aryl, heteroaryl, heterocyclyl, —O(CH₂CH₂O)_{q}—R⁴, —OR⁴, —CN, —C(O)NHR⁴, —C(O)NHS(O)₂R⁴, —NHS(O)₂R⁴, —O—C₂-C₄alkyl-C(O)OR⁴, —C(O)R⁴, —C(O)OR⁴ or carboxylic acid analogs, wherein R⁴ is hydrogen, C₁-C₆ alkyl, C₃-C₈ cycloalkyl, heterocyclyl, aryl or aryl-C₁-C₄ alkyl, or optionally P² is H. Subscript q is from 1 to 6.

In some other embodiments, R¹ is C₁-C₆ alkyl, C₂-C₆ alkenyl or C₃-C₆ cycloalkyl. Each of R² and R³ are independently C₁-C₆ alkyl or C₂-C₆ alkenyl, or R² and R³ are optionally combined to form a C₃-C₆ cycloalkyl. P¹ is a primary pharmacophore of the formula —NH—C(O)—NH—. L is C₁-C₁₂ alkylene, C₃-C₆ cycloalkylene, aryl-C₀-C₆ alkylene, C₃-C₆ cycloalkylene-O-aryl, C₀-C₆alkylenearyl-O-aryl or
C₀-C₆-alkylene-C₃-C₆-heterocycloalkylene. P² is C₂-C₆ alkenyl, C₂-C₆ alkynyl, C₁-C₆ haloalkyl, aryl, heteroaryl, heterocyclyl, —O(CH₂CH₂O)_{q}—R⁴, —OR⁴, —CN, —C(O)NHR⁴, —C(O)NHS(O)₂R⁴, —NHS(O)₂R⁴, —O—C₂-C₄alkyl-C(O)OR⁴, —C(O)R⁴, —C(O)OR⁴ or carboxylic acid analogs, wherein R⁴ is hydrogen, C₁-C₆ alkyl, C₃-C₈ cycloalkyl, heterocyclyl, aryl or aryl-C₁-C₄ alkyl, or optionally P² is H. And subscript q is from 1 to 6.

In other embodiments, the present invention provides a compound wherein R¹ is a member selected from the group consisting of C₁-C₆ alkyl and C₂-C₆ alkenyl; and each of R² and R³ are C₁-C₆ alkyl. In still other embodiments, R¹ is a member selected from the group consisting of isopropyl and isopropenyl.

In another embodiment, the present invention provides a compound wherein L is a member selected from the group consisting of aryl-C₀-C₆ alkyl and C₃-C₆ cycloalkylene-O-aryl. In still another embodiment, L is a member selected from the group consisting of phenyl-C₀-C₆ alkyl and cyclohexylene-O-phenyl. In yet another embodiment, L is phenyl-C₀-C₆ alkyl. In still yet another embodiment, L is cyclohexylene-O-phenyl.

In a further embodiment, the present invention provides a compound wherein P² is a member selected from the group consisting of —CN and —C(O)OR⁴. In other embodiments, R⁴ is a member selected from the group consisting of hydrogen and C₁-C₄ alkyl.

In other embodiments, the present invention provides a compound having Formula Ia:

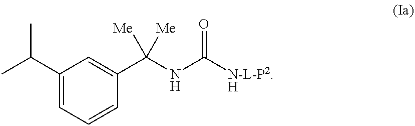

In still other embodiments, the compound of Formula Ia is selected from the group consisting of:

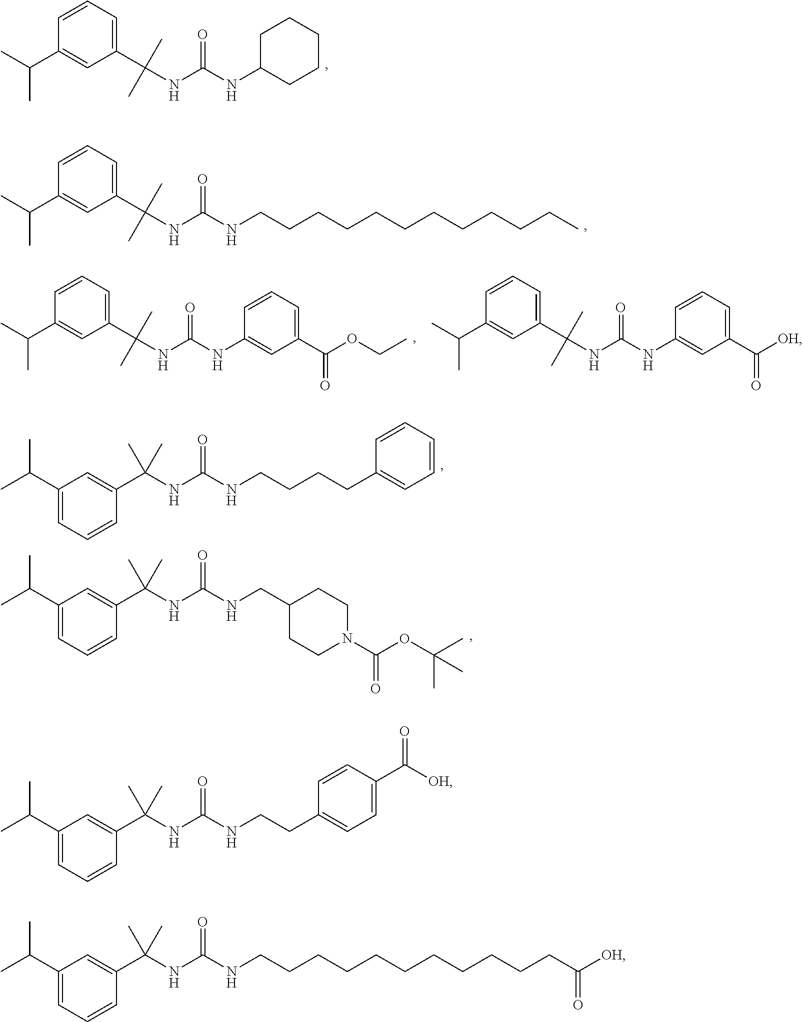

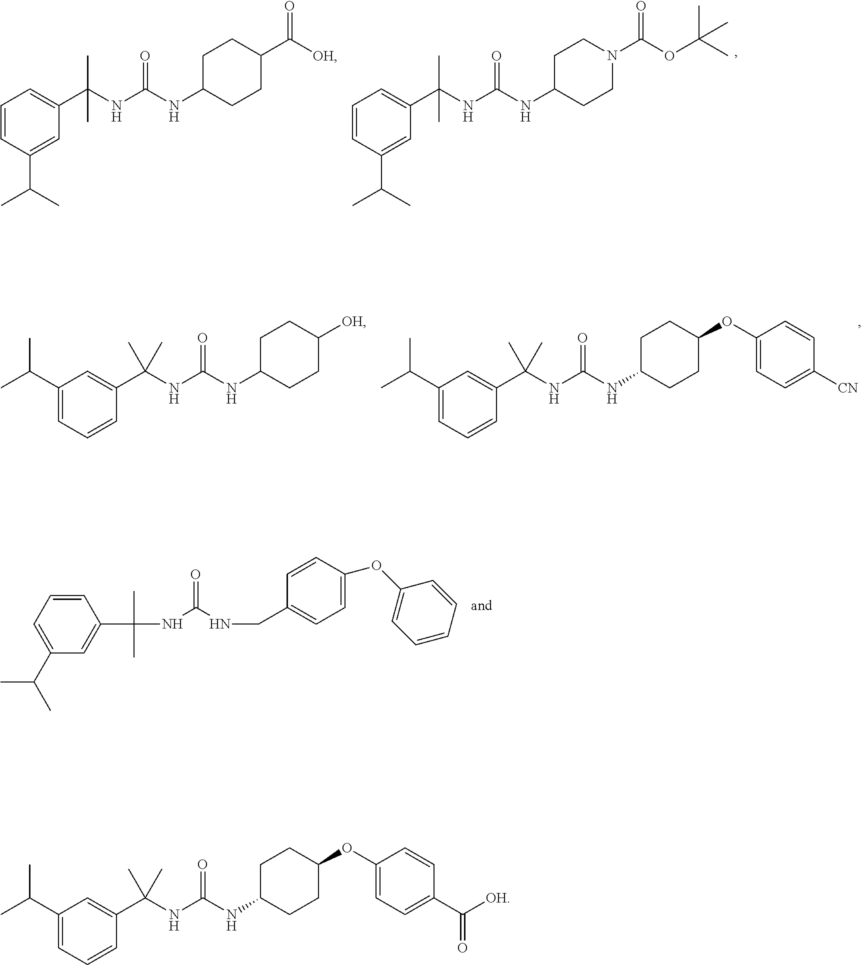

In a further embodiment, the present invention provides a compound having Formula Ib:

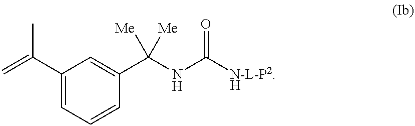

In another embodiment, the compound of Formula Ib is selected from the group consisting of:

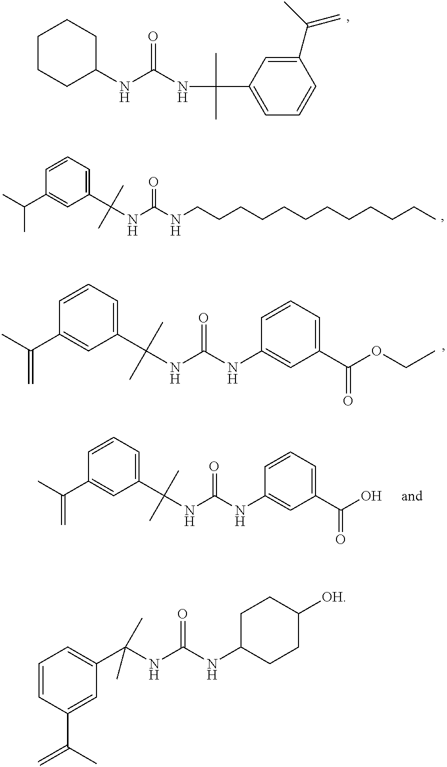

The isoprene group of formula Ib can be modified in a variety of ways, such as by reduction, hydroxylation, epoxidation and halogenation, as well as formation of cyclopropane and cleavage to of the alkene bond to form a ketone. Reduction of the isopropenyl group can be accomplished with hydrogen as well as the isotopes deuterium and tritium. The heavy isotopes are useful for analytical standards or radioactive labels for metabolism and other studies.

TABLE 1
Compounds of the present invention.

| Structure | # |
|---|---|
| 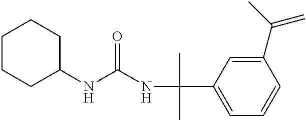 | 1520 |
| 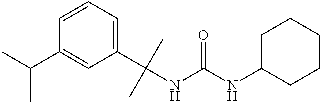 | 1713 |
| 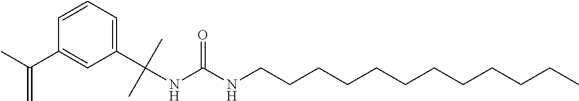 | 1714 |
| 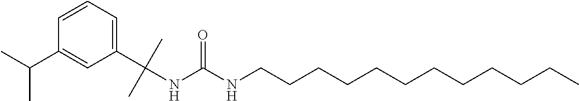 | 1715 |
| 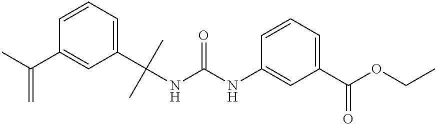 | 1716 |
| 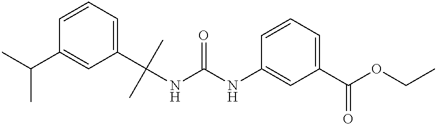 | 1717 |
| 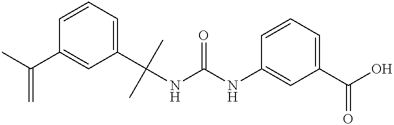 | 1718 |
| 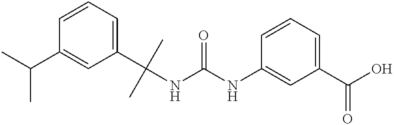 | 1719 |
| 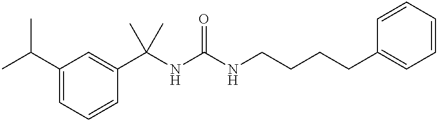 | 1805 |
| 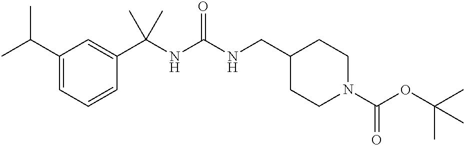 | 1806 |
| 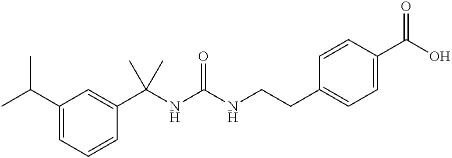 | 1807 |
| 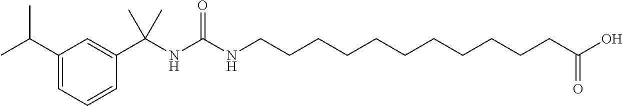 | 1808 |
| 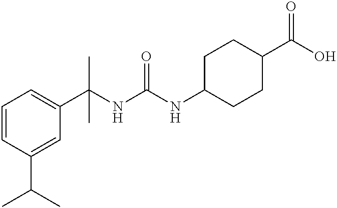 | 1852 |
| 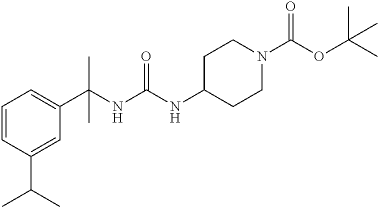 | 1868 |
| 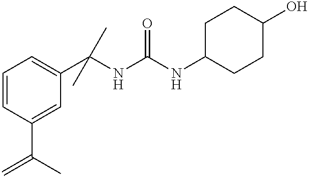 | 1887 |
| 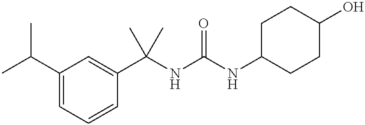 | 1892 |
| 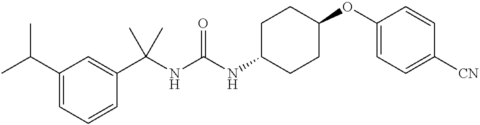 | 1908 |
| 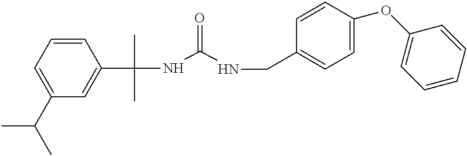 | 1955 |
| 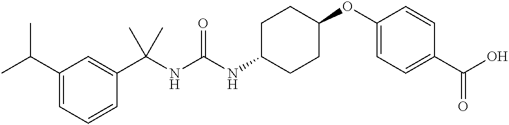 | 1956 |
| 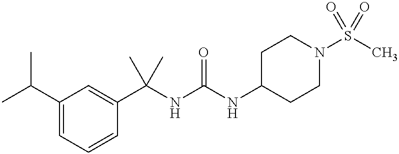 | 2048 |
| 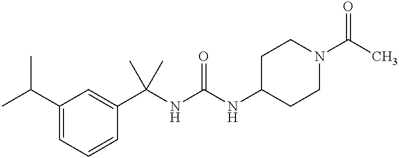 | 2064 |

In one embodiment, sEH inhibitors of the present invention have an IC₅₀ of less than 50 μM for the inhibition of sEH. In another embodiment, the compounds have an IC₅₀ of 1 μM or less. In another embodiment, the compounds have an IC₅₀ of 500 nM or less. In another embodiment, the compounds have an IC₅₀ of 150 nM or less. In another embodiment, the compounds have an IC₅₀ of 100 nM or less. In another embodiment, the compounds have an IC₅₀ of 50 nM or less. In another embodiment, the compounds have an IC₅₀ of 10 nM or less. In another embodiment, the compounds have an IC₅₀ of 1 nM or less.

The compounds of the present invention having an IC₅₀ of less than 500 nM, preferably less than 50 nM, are particularly useful for the treatment of diseases modulated by sEH. Compounds of the present invention having an IC₅₀ of greater than 500 nM can also be useful for the treatment of diseases modulated by sEH, such as by increasing dosage, and for determining structure-activity relationships.

In another embodiment, the present invention provides a pharmaceutical composition comprising a compound of Formula I and a pharmaceutically acceptable excipient. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

A. Methods of Preparation

The compounds of the present invention can be prepared by a variety of methods known to one of skill in the art. In some embodiments, the compounds of the present invention can be prepared as outlined generally in the scheme below.

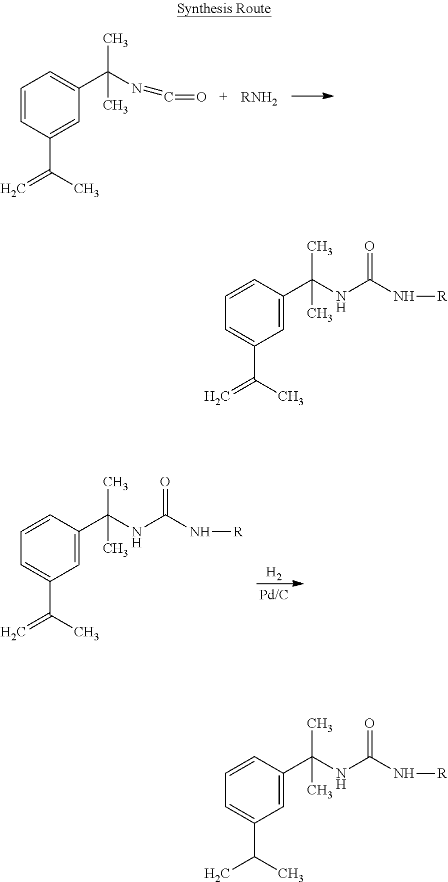

One of skill in the art will appreciate that other methods of preparing the compounds of the present invention are useful in the present invention.

### IV. ASSAYS TO MONITOR SOLUBLE EPOXIDE HYDROLASE ACTIVITY

Additionally, the present invention provides a variety of assays and associated methods for monitoring soluble epoxide hydrolase activity, particularly the activity that has been modulated, such as by inhibiting the activity of the sEH, by the administration of one or more of the compounds provided above.

In one group of embodiments, the invention provides methods for reducing the formation of a biologically active diol produced by the action of a soluble epoxide hydrolase, the method comprising contacting the soluble epoxide hydrolase with an amount of a compound of formula (I) above, sufficient to inhibit the activity of the soluble epoxide hydrolase and reduce the formation of the biologically active diol.

In another group of embodiments, the invention provides methods for stabilizing biologically active epoxides in the presence of a soluble epoxide hydrolase, the method comprising contacting the soluble epoxide hydrolase with an amount of a compound of formula (I), sufficient to inhibit the activity of the soluble epoxide hydrolase and stabilize the biologically active epoxide.

In each of these groups of embodiments, the methods can be carried out as part of an in vitro assay or the methods can be carried out in vivo by monitoring blood titers of the respective biologically active epoxide or diol.

Epoxides and diols of some fatty acids are biologically important chemical mediators and are involved in several biological processes. The strongest biological data support the action of oxylipins as chemical mediators between the vascular endothelium and vascular smooth muscle. Epoxy lipids are anti-inflammatory and anti-hypertensive. Additionally, the lipids are thought to be metabolized by beta-oxidation, as well as by epoxide hydration. Soluble epoxide hydrolase is considered to be the major enzyme involved in the hydrolytic metabolism of these oxylipins. The compounds of formula (I) can inhibit soluble epoxide hydrolase and stabilize the epoxy lipids both in vitro and in vivo. This activity results in a reduction of hypertension in four separate rodent models. Moreover, the inhibitors show a reduction in renal inflammation associated with and independent of the hypertensive models.

More particularly, the present invention provides methods for monitoring a variety of lipids in both the arachidonate and linoleate cascade simultaneously in order to address the biology of the system. A GLC-MS system or a LC-MS method can be used to monitor over 740 analytes in a highly quantitative fashion in a single injection. The analytes include the regioisomers of the arachidonate epoxides (EETs), the diols (DHETs), as well as other P450 products including HETEs. Characteristic products of the cyclooxygenase, lipoxygenase, and peroxidase pathways in both the arachidonate and linoleate series can also be monitored. Such methods are particularly useful as being predictive of certain disease states. The oxylipins can be monitored in mammals following the administration of inhibitors of epoxide hydrolase. Generally, sEH inhibitors increase epoxy lipid concentrations at the expense of diol concentrations in body fluids and tissues.

Other compounds for use in this aspect of the invention are those inhibitors of formula (I) in which the primary pharmacophore is separated from a secondary and/or tertiary pharmacophore by a distance that approximates the distance between the terminal carboxylic acid and an epoxide functional group in the natural substrate.

### V. METHODS OF TREATING DISEASE MODULATED BY SOLUBLE EPOXIDE HYDROLASES

In some embodiments, the present invention provides a method for inhibiting a soluble epoxide hydrolase, comprising contacting said soluble epoxide hydrolase with an inhibiting amount of a compound of Formula I.

In another aspect, the present invention provides methods of treating diseases, especially those modulated by soluble epoxide hydrolases (sEH). The methods generally involve administering to a subject in need of such treatment an effective amount of a compound having a formula (I) above. The dose, frequency and timing of such administering will depend in large part on the selected therapeutic agent, the nature of the condition being treated, the condition of the subject including age, weight and presence of other conditions or disorders, the formulation being administered and the discretion of the attending physician. Preferably, the compositions and compounds of the invention and the pharmaceutically acceptable salts thereof are administered via oral, parenteral, subcutaneous, intramuscular, intravenous or topical routes. Generally, the compounds are administered in dosages ranging from about 2 mg up to about 2,000 mg per day, although variations will necessarily occur depending, as noted above, on the disease target, the patient, and the route of administration. Dosages are administered orally in the range of about 0.05 mg/kg to about 20 mg/kg, more preferably in the range of about 0.05 mg/kg to about 2 mg/kg, most preferably in the range of about 0.05 mg/kg to about 0.2 mg per kg of body weight per day. The dosage employed for topical administration will, of course, depend on the size of the area being treated.

It has previously been shown that inhibitors of soluble epoxide hydrolase (sEH) can reduce hypertension. See, e.g., U.S. Pat. No. 6,351,506. Such inhibitors can be useful in controlling the blood pressure of persons with undesirably high blood pressure, including those who also suffer from diabetes.

In some embodiments, compounds of formula (I) are administered to a subject in need of treatment for hypertension, specifically renal, hepatic, or pulmonary hypertension; inflammation, specifically renal inflammation, vascular inflammation, and lung inflammation; adult respiratory distress syndrome; diabetic complications; end stage renal disease; Raynaud syndrome and arthritis.

### VI. METHODS FOR TREATING DISEASES BY EETS

cis-Epoxyeicosantrienoic acids (“EETs”) can be used in conjunction with the compounds of the invention. EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced.

EETs are well known in the art. EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

EETs produced by the endothelium have anti-hypertensive properties and the EETs 11,12-EET and 14,15-EET may be endothelium-derived hyperpolarizing factors (EDHFs). Additionally, EETs such as 11,12-EET have profibrinolytic effects, anti-inflammatory actions and inhibit smooth muscle cell proliferation and migration. In the context of the present invention, these favorable properties are believed to protect the vasculature and organs during renal and cardiovascular disease states.

It is now believed that sEH activity can be inhibited sufficiently to reduce the hydrolysis of exogenously administered EETs, and thus permit exogenously administered EETs to augment the effects of administering sEH inhibitors by themselves. This permits EETs to be used in conjunction with one or more sEH inhibitors to reduce self-mediated disorders in the methods of the invention. For example, it permits EETs to be used in conjunction with one or more sEH inhibitors to reduce hypertension, or inflammation, or both. Thus, medicaments of EETs can be made which can be administered in conjunction with one or more sEH inhibitors, or a medicament containing one or more sEH inhibitors can optionally contain one or more EETs.

The EETs can be administered concurrently with the sEH inhibitor, or following administration of the sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period during which the inhibitor will be present in amounts to be effective to delay hydrolysis of the EETs. Typically, the EET or EETs will be administered within 48 hours of administering an sEH inhibitor. Preferably, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In some embodiments, the EETs, the compound of the invention, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage the acidic environment of the stomach into the basic environment of the intestines. Such coatings are well known in the art. For example, aspirin coated with so-called “enteric coatings” is widely available commercially. Such enteric coatings may be used to protect EETs during passage through the stomach.

Recent studies from the laboratory of some of the present inventors showed that exogenously administered inhibitors of sEH succeeded in inhibiting sEH sufficiently that levels of EETs could be further raised by the administration of exogenous EETs. Administration of exogenous EETs in conjunction with a sEH inhibitor is therefore expected to be beneficial and to augment the effects of the sEH inhibitor in reducing the progression of diabetic nephropathy.

### VII. METHODS FOR INHIBITING PROGRESSION OF KIDNEY DETERIORATION (NEPHROPATHY) AND REDUCING BLOOD PRESSURE

In another aspect of the invention, the compounds of the invention can reduce damage to the kidney, and especially damage to kidneys from diabetes, as measured by albuminuria. The compounds of the invention can reduce kidney deterioration (nephropathy) from diabetes even in individuals who do not have high blood pressure. The conditions of therapeutic administration are as described above.

The compounds of the present invention can be used with regard to any and all forms of diabetes to the extent that they are associated with progressive damage to the kidney or kidney function. The chronic hyperglycemia of diabetes is associated with long-term damage, dysfunction, and failure of various organs, especially the eyes, kidneys, nerves, heart, and blood vessels. The long-term complications of diabetes include retinopathy with potential loss of vision; nephropathy leading to renal failure; peripheral neuropathy with risk of foot ulcers, amputation, and Charcot joints.

In addition, persons with metabolic syndrome are at high risk of progression to type 2 diabetes, and therefore at higher risk than average for diabetic nephropathy. It is therefore desirable to monitor such individuals for microalbuminuria, and to administer a sEH inhibitor and, optionally, one or more EETs, as an intervention to reduce the development of nephropathy. The practitioner may wait until microalbuminuria is seen before beginning the intervention. As noted above, a person can be diagnosed with metabolic syndrome without having a blood pressure of 130/85 or higher. Both persons with blood pressure of 130/85 or higher and persons with blood pressure below 130/85 can benefit from the administration of sEH inhibitors and, optionally, of one or more EETs, to slow the progression of damage to their kidneys. In some embodiments, the person has metabolic syndrome and blood pressure below 130/85.

Dyslipidemia or disorders of lipid metabolism is another risk factor for heart disease. Such disorders include an increased level of LDL cholesterol, a reduced level of HDL cholesterol, and an increased level of triglycerides. An increased level of serum cholesterol, and especially of LDL cholesterol, is associated with an increased risk of heart disease. The kidneys are also damaged by such high levels. It is believed that high levels of triglycerides are associated with kidney damage. In particular, levels of cholesterol over 200 mg/dL, and especially levels over 225 mg/dL, would suggest that sEH inhibitors and, optionally, EETs, should be administered. Similarly, triglyceride levels of more than 215 mg/dL, and especially of 250 mg/dL or higher, would indicate that administration of sEH inhibitors and, optionally, of EETs, would be desirable. The administration of compounds of the present invention with or without the EETs, can reduce the need to administer statin drugs (HMG-CoA reductase inhibitors) to the patients, or reduce the amount of the statins needed. In some embodiments, candidates for the methods, uses and compositions of the invention have triglyceride levels over 215 mg/dL and blood pressure below 130/85. In some embodiments, the candidates have triglyceride levels over 250 mg/dL and blood pressure below 130/85. In some embodiments, candidates for the methods, uses and compositions of the invention have cholesterol levels over 200 mg/dL and blood pressure below 130/85. In some embodiments, the candidates have cholesterol levels over 225 mg/dL and blood pressure below 130/85.

### VIII. METHODS OF INHIBITING THE PROLIFERATIONS OF VASCULAR SMOOTH MUSCLE CELLS

In other embodiments, compounds of formula (I) inhibit proliferation of vascular smooth muscle (VSM) cells without significant cell toxicity, (e.g., specific to VSM cells). Because VSM cell proliferation is an integral process in the pathophysiology of atherosclerosis, these compounds are suitable for slowing or inhibiting atherosclerosis. These compounds are useful to subjects at risk for atherosclerosis, such as individuals who have had a heart attack or a test result showing decreased blood circulation to the heart. The conditions of therapeutic administration are as described above.

The methods of the invention are particularly useful for patients who have had percutaneous intervention, such as angioplasty to reopen a narrowed artery, to reduce or to slow the narrowing of the reopened passage by restenosis. In some embodiments, the artery is a coronary artery. The compounds of the invention can be placed on stents in polymeric coatings to provide a controlled localized release to reduce restenosis. Polymer compositions for implantable medical devices, such as stents, and methods for embedding agents in the polymer for controlled release, are known in the art and taught, for example, in U.S. Pat. Nos. 6,335,029; 6,322,847; 6,299,604; 6,290,722; 6,287,285; and 5,637,113. In some embodiments, the coating releases the inhibitor over a period of time, preferably over a period of days, weeks, or months. The particular polymer or other coating chosen is not a critical part of the present invention.

The methods of the invention are useful for slowing or inhibiting the stenosis or restenosis of natural and synthetic vascular grafts. As noted above in connection with stents, desirably, the synthetic vascular graft comprises a material which releases a compound of the invention over time to slow or inhibit VSM proliferation and the consequent stenosis of the graft. Hemodialysis grafts are a particular embodiment.

In addition to these uses, the methods of the invention can be used to slow or to inhibit stenosis or restenosis of blood vessels of persons who have had a heart attack, or whose test results indicate that they are at risk of a heart attack.

In one group of embodiments, compounds of the invention are administered to reduce proliferation of VSM cells in persons who do not have hypertension. In another group of embodiments, compounds of the invention are used to reduce proliferation of VSM cells in persons who are being treated for hypertension, but with an agent that is not an sEH inhibitor.

The compounds of the invention can be used to interfere with the proliferation of cells which exhibit inappropriate cell cycle regulation. In one important set of embodiments, the cells are cells of a cancer. The proliferation of such cells can be slowed or inhibited by contacting the cells with a compound of the invention. The determination of whether a particular compound of the invention can slow or inhibit the proliferation of cells of any particular type of cancer can be determined using assays routine in the art.

In addition to the use of the compounds of the invention, the levels of EETs can be raised by adding EETs. VSM cells contacted with both an EET and a compound of the invention exhibited slower proliferation than cells exposed to either the EET alone or to the a compound of the invention alone. Accordingly, if desired, the slowing or inhibition of VSM cells of a compound of the invention can be enhanced by adding an EET along with a compound of the invention. In the case of stents or vascular grafts, for example, this can conveniently be accomplished by embedding the EET in a coating along with a compound of the invention so that both are released once the stent or graft is in position.

### IX. METHODS OF INHIBITING THE PROGRESSION OF OBSTRUCTIVE PULMONARY DISEASE, INTERSTITIAL LUNG DISEASE, OR ASTHMA

Chronic obstructive pulmonary disease, or COPD, encompasses two conditions, emphysema and chronic bronchitis, which relate to damage caused to the lung by air pollution, chronic exposure to chemicals, and tobacco smoke. Emphysema as a disease relates to damage to the alveoli of the lung, which results in loss of the separation between alveoli and a consequent reduction in the overall surface area available for gas exchange. Chronic bronchitis relates to irritation of the bronchioles, resulting in excess production of mucin, and the consequent blocking by mucin of the airways leading to the alveoli. While persons with emphysema do not necessarily have chronic bronchitis or vice versa, it is common for persons with one of the conditions to also have the other, as well as other lung disorders.

Some of the damage to the lungs due to COPD, emphysema, chronic bronchitis, and other obstructive lung disorders can be inhibited or reversed by administering inhibitors of the enzyme known as soluble epoxide hydrolase, or “sEH”. The effects of sEH inhibitors can be increased by also administering EETs. The effect is at least additive over administering the two agents separately, and may indeed be synergistic.

As reported in co-owned U.S. application Ser. No. 10/815,425, EETs can be used in conjunction with sEH inhibitors to reduce damage to the lungs by tobacco smoke or, by extension, by occupational or environmental irritants. These findings indicate that the co-administration of sEH inhibitors and of EETs can be used to inhibit or slow the development or progression of COPD, emphysema, chronic bronchitis, or other chronic obstructive lung diseases which cause irritation to the lungs.

Animal models of COPD and humans with COPD have elevated levels of immunomodulatory lymphocytes and neutrophils. Neutrophils release agents that cause tissue damage and, if not regulated, will over time have a destructive effect. Without wishing to be bound by theory, it is believed that reducing levels of neutrophils reduces tissue damage contributing to obstructive lung diseases such as COPD, emphysema, and chronic bronchitis. As reported in the '425 application, administration of sEH inhibitors to rats in an animal model of COPD resulted in a reduction in the number of neutrophils found in the lungs. Administration of EETs in addition to the sEH inhibitors also reduced neutrophil levels. The reduction in neutrophil levels in the presence of sEH inhibitor and EETs was greater than in the presence of the sEH inhibitor alone.

While levels of endogenous EETs are expected to rise with the inhibition of sEH activity caused by the action of the sEH inhibitor, and therefore to result in at least some improvement in symptoms or pathology, it may not be sufficient in all cases to inhibit progression of COPD or other pulmonary diseases. This is particularly true where the diseases or other factors have reduced the endogenous concentrations of EETs below those normally present in healthy individuals. Administration of exogenous EETs in conjunction with an sEH inhibitor is therefore expected to augment the effects of the sEH inhibitor in inhibiting or reducing the progression of COPD or other pulmonary diseases.

In addition to inhibiting or reducing the progression of chronic obstructive airway conditions, the compounds of the present invention also provide new ways of reducing the severity or progression of chronic restrictive airway diseases. While obstructive airway diseases tend to result from the destruction of the lung parenchyma, and especially of the alveoli, restrictive diseases tend to arise from the deposition of excess collagen in the parenchyma. These restrictive diseases are commonly referred to as “interstitial lung diseases”, or “ILDs”, and include conditions such as idiopathic pulmonary fibrosis. The methods, compositions and uses of the invention are useful for reducing the severity or progression of ILDs, such as idiopathic pulmonary fibrosis. Macrophages play a significant role in stimulating interstitial cells, particularly fibroblasts, to lay down collagen. Without wishing to be bound by theory, it is believed that neutrophils are involved in activating macrophages, and that the reduction of neutrophil levels found in the studies reported herein demonstrate that the methods and uses of the invention will also be applicable to reducing the severity and progression of ILDs.

In some embodiments, the ILD is idiopathic pulmonary fibrosis. In other embodiments, the ILD is one associated with an occupational or environmental exposure. Exemplars of such ILDs, are asbestosis, silicosis, coal worker's pneumoconiosis, and berylliosis. Further, occupational exposure to any of a number of inorganic dusts and organic dusts is believed to be associated with mucus hypersecretion and respiratory disease, including cement dust, coke oven emissions, mica, rock dusts, cotton dust, and grain dust (for a more complete list of occupational dusts associated with these conditions, see Table 254-1 of Speizer, “Environmental Lung Diseases,” Harrison's Principles of Internal Medicine, infra, at pp. 1429-1436). In other embodiments, the ILD is sarcoidosis of the lungs. ILDs can also result from radiation in medical treatment, particularly for breast cancer, and from connective tissue or collagen diseases such as rheumatoid arthritis and systemic sclerosis. It is believed that the methods, uses and compositions of the invention can be useful in each of these interstitial lung diseases.

In another set of embodiments, the compounds of the present invention are used to reduce the severity or progression of asthma. Asthma typically results in mucin hypersecretion, resulting in partial airway obstruction. Additionally, irritation of the airway results in the release of mediators which result in airway obstruction. While the lymphocytes and other immunomodulatory cells recruited to the lungs in asthma may differ from those recruited as a result of COPD or an ILD, it is expected that the invention will reduce the influx of immunomodulatory cells, such as neutrophils and eosinophils, and ameliorate the extent of obstruction. Thus, it is expected that the administration of sEH inhibitors of the present invention, and the administration of sEH inhibitors of the present invention in combination with EETs, will be useful in reducing airway obstruction due to asthma.

In each of these diseases and conditions, it is believed that at least some of the damage to the lungs is due to agents released by neutrophils which infiltrate into the lungs. The presence of neutrophils in the airways is thus indicative of continuing damage from the disease or condition, while a reduction in the number of neutrophils is indicative of reduced damage or disease progression. Thus, a reduction in the number of neutrophils in the airways in the presence of an agent is a marker that the agent is reducing damage due to the disease or condition, and is slowing the further development of the disease or condition. The number of neutrophils present in the lungs can be determined by, for example, bronchoalveolar lavage.

### X. PROPHYLACTIC AND THERAPEUTIC METHODS TO REDUCE STROKE DAMAGE

Inhibitors of soluble epoxide hydrolase (“sEH”) and EETs administered in conjunction with inhibitors of sEH have been shown to reduce brain damage from strokes (see U.S. Published Application No. 2006/0148744). Based on these results, we expect that compounds of the present invention taken prior to an ischemic stroke will reduce the area of brain damage and will likely reduce the consequent degree of impairment. The reduced area of damage should also be associated with a faster recovery from the effects of the stroke.

While the pathophysiologies of different subtypes of stroke differ, they all cause brain damage. Hemorrhagic stroke differs from ischemic stroke in that the damage is largely due to compression of tissue as blood builds up in the confined space within the skull after a blood vessel ruptures, whereas in ischemic stroke, the damage is largely due to loss of oxygen supply to tissues downstream of the blockage of a blood vessel by a clot. Ischemic strokes are divided into thrombotic strokes, in which a clot blocks a blood vessel in the brain, and embolic strokes, in which a clot formed elsewhere in the body is carried through the blood stream and blocks a vessel there. But, in both hemorrhagic stroke and ischemic stroke, the damage is due to the death of brain cells. The compounds of the present invention are expected to provide at least some reduction in brain damage in all types of stroke and in all subtypes.

A number of factors are associated with an increased risk of stroke. sEH inhibitors administered to persons with any one or more of the following conditions or risk factors: high blood pressure, tobacco use, diabetes, carotid artery disease, peripheral artery disease, atrial fibrillation, transient ischemic attacks (TIAs), blood disorders such as high red blood cell counts and sickle cell disease, high blood cholesterol, obesity, alcohol use of more than one drink a day for women or two drinks a day for men, use of cocaine, a family history of stroke, a previous stroke or heart attack, or being elderly, will reduce the area of brain damaged by a stroke. With respect to being elderly, the risk of stroke increases for every 10 years. Thus, as an individual reaches 60, 70, or 80, administration of sEH inhibitors has an increasingly larger potential benefit. The administration of EETs in combination with one or more sEH inhibitors (sEHI) of the present invention can be beneficial in further reducing the brain damage. One can expect beneficial effects from sEHI with or without EETs in a variety of diseases which lead to ischemia reperfusion injury, such as heart attacks.

In some uses and methods, the sEH inhibitors and, optionally, EETs, are administered to persons who use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack, or are 60, 70, or 80 years of age or more.

Clot dissolving agents, such as tissue plasminogen activator (tPA), have been shown to reduce the extent of damage from ischemic strokes if administered in the hours shortly after a stroke. tPA, for example, is approved by the FDA for use in the first three hours after a stroke. Thus, at least some of the brain damage from a stroke is not instantaneous, but occurs over a period of time or after a period of time has elapsed after the stroke. It is therefore believed that administration of sEH inhibitors, optionally with EETs, can also reduce brain damage if administered within 6 hours after a stroke has occurred, more preferably within 5, 4, 3, or 2 hours after a stroke has occurred, with each successive shorter interval being more preferable. Even more preferably, the inhibitor or inhibitors are administered 2 hours or less or even 1 hour or less after the stroke, to maximize the reduction in brain damage. Persons of skill are well aware of how to make a diagnosis of whether or not a patient has had a stroke. Such determinations are typically made in hospital emergency rooms, following standard differential diagnosis protocols and imaging procedures.

In some uses and methods, the sEH inhibitors and, optionally, EETs, are administered to persons who have had a stroke within the last 6 hours who: use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

The conditions of therapeutic administration for all of these indications are as described above.

### XI. COMBINATION THERAPY

As noted above, the compounds of the present invention will, in some instances, be used in combination with other therapeutic agents to bring about a desired effect. Selection of additional agents will, in large part, depend on the desired target therapy (see, e.g., Turner, N. et al. Prog. Drug Res. (1998) 51: 33-94; Haffner, S. Diabetes Care (1998) 21: 160-178; and DeFronzo, R. et al. (eds.), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W., (ed.), Current Therapy In Endocrinology And Metabolism, 6th Edition (Mosby—Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). Combination therapy includes administration of a single pharmaceutical dosage formulation which contains a compound having the general structure of formula 1 and one or more additional active agents, as well as administration of a compound of formula 1 and each active agent in its own separate pharmaceutical dosage formulation. For example, a compound of formula 1 and one or more angiotensin receptor blockers, angiotensin converting enzyme inhibitors, calcium channel blockers, diuretics, alpha blockers, beta blockers, centrally acting agents, vasopeptidase inhibitors, renin inhibitors, endothelin receptor agonists, AGE crosslink breakers, sodium/potassium ATPase inhibitors, endothelin receptor agonists, endothelin receptor antagonists, angiotensin vaccine, and the like; can be administered to the human subject together in a single oral dosage composition, such as a tablet or capsule, or each agent can be administered in separate oral dosage formulations. Where separate dosage formulations are used, a compound of formula 1 and one or more additional active agents can be administered at essentially the same time (i.e., concurrently), or at separately staggered times (i.e., sequentially). Combination therapy is understood to include all these regimens.

The following examples are provided to illustrate the invention and are not intended to limit any aspect of the invention as set forth above or in the claims below.

### XII. EXAMPLES

All melting points were determined with a Thomas-Hoover apparatus (A. H. Thomas Co.) and are uncorrected. Compounds with no melting point values exist in the solid state as either foams or glassy solids. Mass spectra were measured by LC-MS (Waters 2790). ¹H-NMR spectra were recorded on QE-300 spectrometer, using tetramethylsilane as an internal standard. Signal multiplicities are represented as singlet (s), doublet (d), double doublet (dd), triplet (t), quartet (q), quintet (quint), multiplet (m), broad (br), broad singlet (brs), broad doublet (br d), broad triplet (br t), broad multiplet (br m), doublet of doublet of doublets (ddd) and quartet of doublets (qd). Synthetic methods are described for representative compounds.

The abbreviations used in the examples below have the following meaning: melting point (Mp), mass spectroscopy (MS), thin layer chromatography (TLC), the parent peak in the MS plus H⁺ ([M+H]⁺), minute (min), kilogram (kg), milligram (mg), nanomolar (nM), tetrahydrofuran (THF), tertiary butoxy carbonyl (BOC), potassium sulfate (KHSO₄), potassium hydroxide (KOH), magnesium sulfate (MgSO₄), hydrogen chloride (HCl), dimethylsulfoxide (DMSO), ethyl (Et), ethyl acetate (EtOAc), methanol (MeOH), dichloromethane (CH₂Cl₂, DCM), area under the concentration (AUC).

Lower case bolded Roman numerals in the examples below refer to the corresponding intermediates in Scheme 1 above. Compounds numbers are also used as provided in the Schemes as well as in the Tables below.

### Example 1

### Preparation of 1-cyclohexyl-3-(2-(3-prop-1-en-2-yl)phenyl)propan-2-yl)urea (1520)

400 mg 1-(2-isocyantopropane-2yl)-3-(prop-1-en2-yl)benzene and 220 mg of cyclohexylamine were added to 5 ml toluene. The mixture was heated to reflux and cooled to yield 300 mg of 1-cyclohexyl-3-(2-(3-prop-1-en-2yl)phenyl)propan-2-yl)urea. ¹H NMR Shifts (ppm, δ) (CDCl₃): 7.60 (s, 1H, aryl), 7.20-7.40 (m, 3H, aryl), 6.38 (s, 1H, HC═C), 6.10 (s, 1H, HC═C), 4.78 (s, 1H, NH), 3.98 (d, 1H, NH), 3.52 (m, 1H, NHCH), 2.28 (s, 3H, CH3C═), 0.85-1.50 (m, 10H, cyclohexyl).

### Example 2

### Preparation of 1-cyclohexyl-3-(2-(3-isopropylphenyl)propan-2-yl)urea (1713)

100 mg of 1-cyclohexyl-3-(2-(3-prop-1-en-2-yl)phenyl)propan-2-yl)urea were placed in 10 ml absolute ethanol and a few mg of 10% Pd on carbon were added. Hydrogen was introduced over this mixture. After 24 hrs the catalyst was filtered off and removal of the solvent provided 75 mg of 1-cyclohexyl-3-(2-(3-isopropylphenyl)propan-2-yl)urea. ¹H NMR Shifts (ppm, δ) (D₆DMSO): 7.05-7.25 (m, 4H, aryl), 6.10 (s, 1H, NH), 5.78 (d, 1H, NH), 3.28 (m, 1H, NHCH), 2.83 (m,1H, CH(CH3)₂), 1.62 (m, 5H, cyclohexyl), 1.50 (s, 6H, CH₃)₂C), 1.0-1.38 (m, 5H, cyclohexyl), 1.20 (d, 6H, (CH₃)₂CH).

### Example 3

### Preparation of Compounds 1714-1719, 1805-1808, 1852 and 1868

Preparation of additional compounds 1714-1719, 1805-1808, 1852 and 1868 was accomplished using the methods described above using appropriate starting materials. ¹H NMR Shifts are provided in the table below.

TABLE 2
Table of ¹H NMR shifts for compounds 1714-1719, 1805-1808, 1852 and 1868.

| Inhibitor | Solvent | ¹H NMR Shifts (ppm, δ) |
|---|---|---|
| 1714 | CDCl₃ | 7.60 (s, 1H, aryl), 7.30-7.42 (m, 3H, aryl), 5.38 (s, 1H, HC═C), |
| | | 5.17 (s, 1H, HC═C), 4.78 (s, 1H, NH), |
| | | 3.97 (m, 1H, NH), 3.02 (q, 1H, NHCH ₂), 2.17 (s, 3H, CH₃C═), |
| | | 1.63 (s, 6H, CH₃)₂C), 1.00-1.40 (m, 22, CH₂), 0.85 (t, 3, CH₃) |
| 1715 | CDCl₃ | 7.18-7.40 (m, 4H, aryl), 4.78 (s, 1H, NH), 3.95 (t, 1H, NH), |
| | | 3.00 (m, 1H, NHCH₂), 3.05 (m, 1H, CH(CH3)₂), |
| | | 1.60 (s, 6H, (CH₃)₂C), 0.90-1.40 (m. 22H, CH₂), 0.82 (t, 3H, |
| | | CH₃) |
| 1716 | D₆DMSO | 8.66 (s, 1H, aryl) 8.0 (m 1H, aryl), 7.40-7.50 (m, 3H, aryl), |
| | | 7.22-7.33 (m, 4H, aryl, NH), 6.60 (s, 1H, NH) 5.35 (s, 1H, |
| | | HC═C), 5.05 (s, 1H, HC═C), 4.24 (q, 2H, OCH₂), 2.08 (s, 3H, |
| | | CH₃C═C), 1.58 (s, 6H, C(CH₃)₂), 1.26 (t, 3H, CH₃CH₂) |
| 1717 | D₆DMSO | 8.68 (s, 1H, aryl), 8.00 (m, 1H, aryl), 7.06-7.50 (m, 7H, aryl, |
| | | NH), 6.58 (s, 1H, NH), 4.28 (q, 2H, OCH₂), |
| | | 2.88 (m, 1H, CH(CH₃)₂), 1.60 (s, 6H, CH3)₂C), 1.30 (t, 3H, |
| | | CH₃CH₂), 1.20 (d, 6H, CH₃)₂CH) |
| 1718 | D₆DMSO | 12.85 (s, 1H, OH), 8.23 (s, 1H, aryl), 8.05 (m, 1H, aryl), |
| | | 7.63-7.55 (m, 7H, aryl, NH), 6.72 (s, 1H, NH) 5.38 (s, 1H, HC═C), |
| | | 5.08 (s, 1H, HC═C), 2.13 (s, 3H, CH₃C═), 1.63 (s, 6H, |
| | | C(CH3)₂) |
| 1719 | D₆DMSO | 12.80 (s, 1H, OH), 8.40 (s, 1H, aryl), 8.20 (m, 1H, aryl), |
| | | 7.08-7.48 (m, 7H, aryl, NH), 2.70 (m, 1, CH(CH₃)₂), 1.60 (s, 6H, |
| | | C(CH3)₂ 1.20 (d, 6H, CH₃)₂CH) |
| 1805 | CDCl₃ | 7.06-7.36 (m, 9H, aryl), 4.72 (s, 1H, NH), 3.88 (m, 1H, NH), |
| | | 3.05 (m, 2H, CH₂N), 2.88 (m, 1H, HC(CH₃)₂), 2.48 (t, 2H, |
| | | CH₂), 1.60 (s, 6H, C(CH₃)₂), 1.22 (d, 6H, CH(CH₃)₂) |
| 1806 | CDCl₃ | 7.15-7.40 (m, 4H, aryl), 4.80 (s, 1H, NH), 4.00 (t, 3H, CH2N, |
| | | pip. ring), 2.95 (m, 3H, piper. ring), 2.58 (m, 1H, NHCH), |
| | | 2.58 (m, 1H, HC(CH₃)₂), 1.60 (s, 6H, C(CH₃)₂), 1.52 (s, 9H, |
| | | C(CH₃)₃), 1.25 (d, 6, CH(CH₃)₂) |
| 1807 | D₆DMSO | 12.84 (s, 1H, OH), 7.90 (m, 2H, aryl), 7.00-7.2 (m, 6H, aryl), |
| | | 6.27 (s, 1H, NH), 5.82 (m, 1H, NH), 3.32 (m, 2H, CH₂N), |
| | | 2.89 (m, 1H, CH(CH₃)₂), 2.74 (t, 2H, CH₂), 1.51 (s, 6H, C(CH₃)₂), |
| | | 1.20 (d, 6H, CH(CH₃)₂) |
| 1808 | CDCl₃ | (m, 4H, aryl), 5.88 (s, 1H, NH), 2.94 (m, 3H, NCH₂, |
| | | HC(CH₃)₂), 2.36 (t, 2H, CH₂CO₂H), 1.64 (s, 6H, C(CH₃)₂), |
| | | 1.10-1.40 (m, 22H, CH₂, CH(CH₃)₂) |
| 1852 | D₆DMSO | 12.10 (s, 1H, CO₂H), 7.10 (m, 3H, aryl) 7.00 (d, 1H, aryl), |
| | | 6.16 (s, 1H, NH), 5.95 (d, NH), 3.30-3.52 (m, H, cyclohex. ring), |
| | | 2.84 (m, 2H, NCH₂), 2.25 (m, 1H, CH(CO₂H), 1.63 (m, H, |
| | | cyclohex ring, HC(CH₃)₂), 1.48 (s, 6H, C(CH₃)₂), |
| | | 1.16 (d, CH(CH₃)₂) |
| 1868 | D₆DMSO | 7.05-7.22 (m, 4H, aryl), 6.17 (s, 1H, NH), 5.82 (d, 1H, NH), |
| | | 3.75 (m, H, piper. ring), 3.42 (m, H, piper. ring), 2.84 (m, piper. |
| | | ring), 1.50 (s, 6H, C(CH₃)₂), 1.39 (s, 9H, C(CH₃)₃), 1.20 (d, 6H, |
| | | CH(CH₃)₂) |
| 1995 | D₆DMSO | 7.42-6.50 (m, 13H, aryl), 6.36 (s, 1H, NH), 6.26 (t, 1H, |
| | | NHCH₂aryl), 4.13 (d, 2H, NHCH₂aryl), 2.81 (m, 1H, |
| | | CH(CH₃)₂, 1.53 (s, 6H, CH₃), 1.16 (d, 6H, CH(CH₃)₂) |

NMR spectra were recorded on a Varian Mercury 300 (Varian Inc., Palo Alto, Calif.) in CDCl₃ or DMSO-d6 using tetramethylsilane (TMS) as an internal reference unless otherwise noted. NMR peaks are reported in parts per million (ppm, δ) relative to TMS.

### Example 4

### Preparation of trans-1-(4-Hydroxy-cyclohexyl)-3-[1-(3-isopropenyl)-1-methyl-ethyl]-urea (1887)

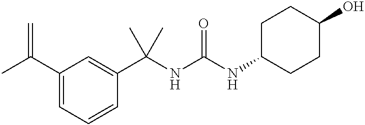

To a solution of 3-isopropenyl-alpha,alpha-dimethylbenzyl isocyanate (3.0 g, 15 mmol) in DMF (75 mL) were added trans-4-aminocyclohexanol hydrochloride (3.4 g, 22 mmol) and Et₃N (3.1 mL, 22 mmol) at 0° C. The reaction mixture was warmed up to room temperature and stirred overnight. After adding IN HCl (40 mL) and water, the resulting white precipitates were collected by suction filtration. The collected solid was thoroughly washed with water. Recrystallization from methanol afforded 4.2 g (89%) of the title compound as a white solid. ¹H NMR (300 MHz, DMSO-d₆): δ 7.43 (s, 1H), 7.31-7.22 (m, 3H), 6.14 (s, 1H), 5.68 (d, J=8 Hz, 1H), 5.36 (s, 1H), 5.09-5.06 (m, 1H), 4.48 (d, J=4 Hz, 1H), 3.42-3.30 (m, 1H), 3.26-3.11 (m, 1H), 2.09 (s, 3H), 1.80-1.66 (m, 4H), 1.51 (s, 6H), 1.22-0.96 (m, 4H).

### Example 5

### Preparation of trans-1-[4-(4-Cyano-phenoxy)-cyclohexyl]-3-[1-(3-isopropyl-phenyl)-1-methyl-ethyl]-urea (1908)

### Preparation of trans-1-[4-(4-Cyano-phenoxy)-cyclohexyl]-3-[1-(3-isopropenyl-phenyl)-1-methyl-ethyl]-urea

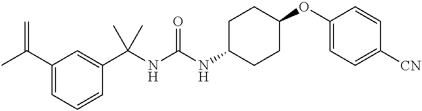

To a solution of 1887 (1.0 g, 3.2 mmol) in DMF (32 mL) was added 60% sodium hydride in oil (0.19 g, 4.74 mmol) at 0° C. After 10 min, 4-fluorobenzonitrile (0.5 g, 4.2 mmol) was added. The reaction mixture was allowed to slowly warm to room temperature overnight. The reaction was quenched by adding water and the resulting white precipitates were collected and washed with water. Purification by column chromatography (6:4 Hexanes-EtOAc) gave the title compound, 1.18 g (88%) as a white solid. ¹H NMR (300 MHz, DMSO-d₆) δ 7.73 (d, J=9 Hz, 2H), 7.45-7.43 (m, 1H), 7.31-7.23 (m, 3H), 7.11 (d, J=9 Hz, 2H), 6.17 (s, 1H), 5.82 (d, J=8 Hz, 1H), 5.36 (s, 1H), 5.09-5.06 (m, 1H), 4.54-4.41 (m, 1H), 3.39-3.27 (m, 1H), 2.10 (s, 3H), 2.05-1.94 (m, 2H), 1.87-1.77 (m, 2H), 1.53 (s, 6H), 1.49-1.35 (m, 2H), 1.33-1.16 (m, 2H).

### Preparation of trans-1-[4-(4-Cyano-phenoxy)-cyclohexyl]-3-[1-(3-isopropyl-phenyl)-1-methyl-ethyl]-urea (1908)

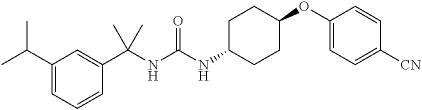

To a solution of the above compound (1.0 g, 2.4 mmol) in EtOAc was added 10% palladium on carbon. The solution was filled with H₂ and the reaction mixture was stirred overnight. After the solution was filtered through Celite, the filtrate was concentrated in vacuo. Purification by column chromatography (6:4 Hexanes-EtOAc) gave the title compound, 0.6 g (60%) as a white solid. ¹H NMR (400 MHz, CDCl₃): δ 7.74 (d, J=9 Hz, 2H), 7.22-7.02 (m, 6H), 6.11 (s, 1H), 5.80 (d, J=8 Hz, 1H), 4.52-4.44 (m, 1H), 3.37-3.26 (m, 1H), 2.84 (septet, J=7 Hz, 1H), 2.04-1.95 (m, 2H), 1.86-1.78 (m, 2H), 1.51 (s, 6H), 1.46-1.22 (m, 4H), 1.19 (d, J=7 Hz, 6H).

### Example 6

### Preparation of trans-4-(4-{3-[1-Isopropyl-phenyl)-1-methyl-ethyl]-ureido}-cyclohexyloxy]-benzoic acid (1956)

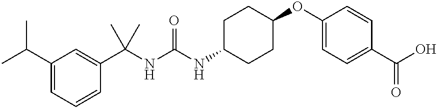

To a solution of 1908 (0.5 g, 1.2 mmol) in EtOH was added 4 mL of 6N NaOH at room temperature. The reaction mixture was refluxed overnight. The solvent was evaporated in vacuo and washed with EtOAc. The aqueous layer was acidified with 1N HCl to give white precipitates. The resulting white solids were collected by suction filtration and washed with water. Purification by column chromatography (3:7 Haxanes-EtOAc) gave the title compound, 0.26 g (50%) as a white solid. ¹H NMR (300 MHz, DMSO-d₆) δ 12.59 (s, 1H), 7.85 (d, J=9 Hz, 2H), 7.22-7.10 (m, 3H), 7.06-6.98 (m, 3H), 6.12 (s, 1H), 5.80 (d, J=8 Hz, 1H), 4.50-4.38 (m, 1H), 3.41-3.26 (m, 1H), 2.84 (septet, J=7 Hz, 1H), 2.07-1.95 (m, 2H), 1.88-1.76 (m, 2H), 1.51 (s, 6H), 1.46-1.22 (m, 4H), 1.19 (d, J=7 Hz, 6H).

### Example 7

### Inhibition of Human Soluble Epoxide Hydrolase

This example provides assays and illustrates the inhibition of human soluble epoxide hydrolases by compounds of the invention.

Enzyme Preparation

Recombinant human sEH was produced in a baculovirus expression system and purified by affinity chromatography. The preparations were at least 97% pure as judged by SDS-PAGE and scanning densitometry. No detectable esterase or glutathione transferase activity, which can interfere with this sEH assay, was observed. Protein concentration was quantified by using the Pierce BCA assay using Fraction V bovine serum albumin as the calibrating standard.

IC₅₀ Assay Conditions

IC₅₀ values were determined in one of three methods. One method uses racemic 4-nitrophenyl-trans-2,3-epoxy-3-phenylpropyl carbonate as substrate. Enzyme (0.24 μM human sEH) was incubated with inhibitors for 5 min in sodium phosphate buffer, 0.1 M pH 7.4, at 30° C. before substrate introduction([S]=40 μM). Activity was assessed by measuring the appearance of the 4-nitrophenolate anion at 405 nm at 30° C. during 1 min (Spectramax 200; Molecular Devices). Assays were performed in triplicate. 10₅₀ is a concentration of inhibitor, which reduces enzyme activity by 50%, and was determined by regression of at least five datum points with a minimum of two points in the linear region of the curve on either side of the IC₅₀. The curve was generated from at least three separate runs, each in triplicate.

Other IC₅₀ values were determined using the procedure described in Analytical

Biochemistry 343 66-75 (2005) using cyano(6-methoxy-naphthalen-2-yl)methyl trans-[(3-phenyloxiran-2-yl)methyl]carbonate as a substrate. Enzymes (0.96 nM for human sEH) were incubated with inhibitors ([I]=0.5-10,000 nM) for 5 min in BisTris-HCl buffer (25 mM, pH 7.0, containing 0.1 mg/ml of BSA) at 30° C. prior to substrate introduction ([S]=5 lM). Enzyme activity was measured by monitoring the appearance of 6-methoxy-2-naphthaldehyde. Assays were performed in triplicate. By definition, IC₅₀ values are concentrations of inhibitor that reduce enzyme activity by 50%. IC₅₀ values were determined by regression of at least five datum points, with a minimum of two datum points in the linear region of the curve on either side of the IC₅₀ values. The curve was generated from at least three separate runs, each in triplicate.

Other inhibition potencies were determined using a fluorescent based high-throughput assay. Inhibitors in solution at 10 mM in DMSO were serially diluted by 10-fold increment in Bis/Tris HCl buffer (25 mM pH 7.0) containing 0.1 mg/mL of BSA (Buffer A). In black 96-well plates, 204 of the inhibitor dilution or buffer were delivered in every well, and then 130 μL of Human sEH at ˜0.4 μg/mL in solution in Buffer A were added to each well. The plate was then mixed and incubated at room temperature for 5 minutes. Fifty microliters of substrate ((3-Phenyl-oxiranyl)-acetic acid cyano-(6-methoxy-naphthalen-2-yl)-methyl ester; PHOME) at 200 μM in solution in 96:4 Buffer A:DMSO was then added to each well to give [S]final=50 μM and [E]final=˜4 nM. The plate was then mixed and incubated in the dark at room temperature (˜25° C.) for 90 min. Activity was measured by determining the relative quantity of 6-methoxy-2-naphthaldehyde formed with an excitation wavelength of 316 nm and an emission wavelength of 460 nm measured with a SpectraMax M-2 fluorometer (molecular Devices, Sunnyvale, Calif.).

Assays were conducted with the compounds indicated in the table below, as described above.

TABLE 3
IC₅₀ data.

| | | Human |
|---|---|---|
| | | sEH |
| Structure | Compound | IC₅₀ (nM) |
| 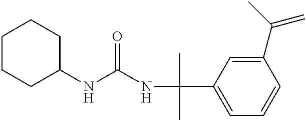 | 1520 | 11.4 |
| 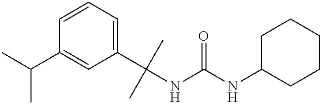 | 1713 | 7.5 |
| 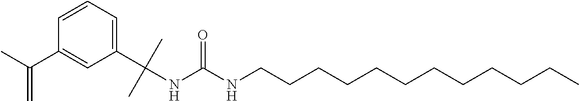 | 1714 | 33 |
| 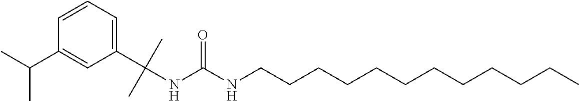 | 1715 | 10.9 |
| 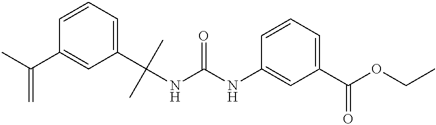 | 1716 | 41.3 |
| 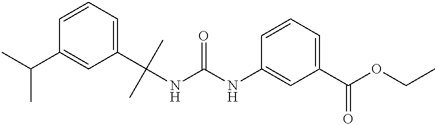 | 1717 | 157.9 |
| 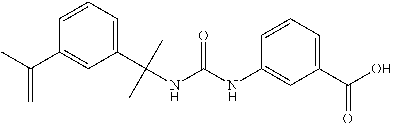 | 1718 | 7919 |
| 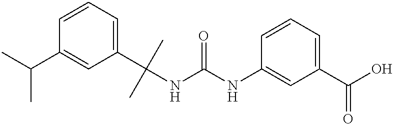 | 1719 | 1609.7 |
| 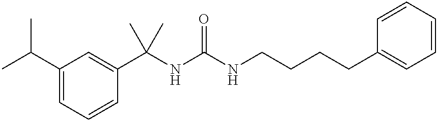 | 1805 | 11.5 |
| 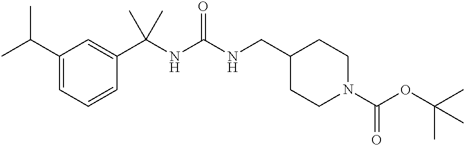 | 1806 | 5.6 |
| 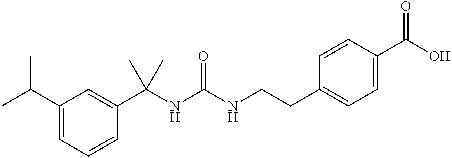 | 1807 | 2888.1 |
| 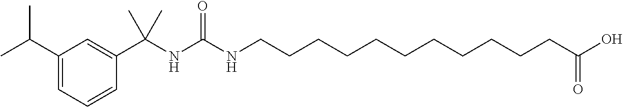 | 1808 | 52.5 |
| 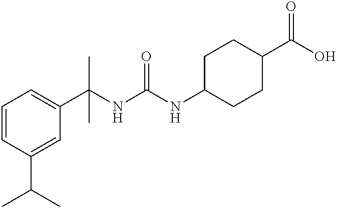 | 1852 | 554.9 |
| 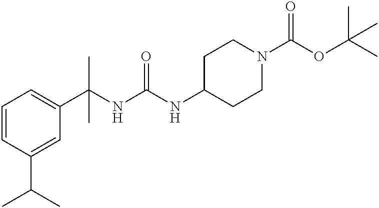 | 1868 | 21.6 |
| 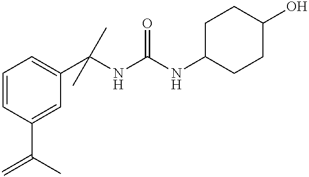 | 1887 | 76.7 |
| 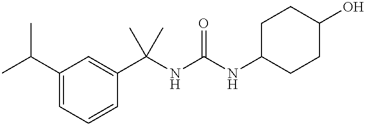 | 1892 | 392.7 |
| 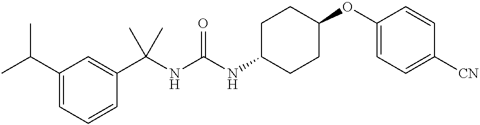 | 1908 | 9.8 |
| 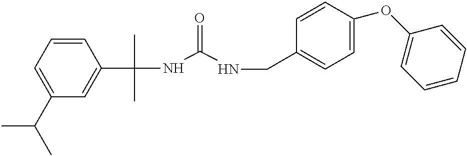 | 1955 | 10.6 |
| 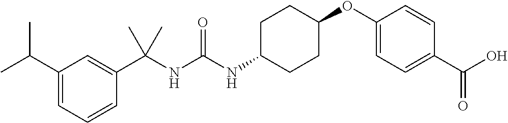 | 1956 | 3.9 |
| 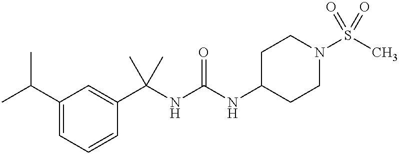 | 2048 | 97.6 |
| 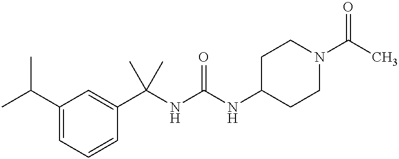 | 2064 | 444 |

### Example 8

### In Vivo Inhibition of Soluble Epoxide Hydrolase

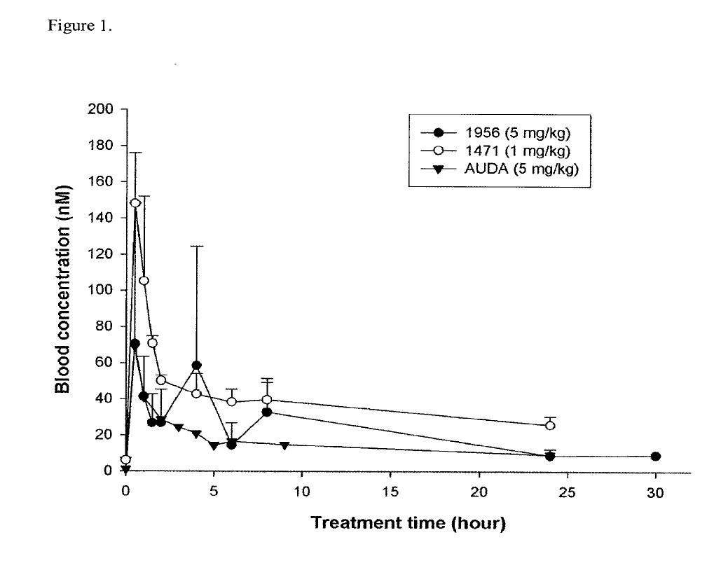

This example provides the pharmacokinetic studies carried out using compounds of the present invention. See FIG. 1.

The pharmacokinetic properties of some of the most potent sEH inhibitors was evaluated following oral gavage in mice. As noted above, the use of 1-adamantyl urea inhibitors afforded exquisite sensitivity, allowing the determination of the determined pharmacokinetic parameters from serial blood samples collected from individual mice (see Table 17).

Animals. Male Swiss Webster mice, 6 weeks-old, were obtained from Charles River (CA, USA). After 1-2 week acclamation period, healthy animals were assigned to study groups based on body-weight stratified randomization procedure. The body weight of animals used in all the experiments ranged from 28 g to 38 g. Mice were maintained on a 12 h light/12 h dark cycle under controlled temperature and humidity conditions, and food and water available ad libid um.

Administration and measurement. Pharmacokinetic studies in mice used a 1 or 5 mg/kg dose of sEH inhibitors 1956, trans-4-(4-{1-adamantyl-ureido}-cyclohexyloxy)-benzoic acid (1471), and 1-(1-Adamantyl)-3-(dodecanoic acid)urea (AUDA) dissolved in corn oil and 4% DMSO administered orally. Serial tail bled blood samples (5-10 μL)were collected in heparinized 1.5 mL tubes at various time points (0.5, 1, 2, 3, 4, 5, 6, and 24 hr) after the administration for measuring parent compounds and their metabolites by using LC-MS/MS: a Waters 2790 liquid chromatograph equipped with a 30×2.1 mm 3 μm C18 Xterra™ column (Waters) and a Micromass Quattro Ultima triple quadrupole tandem mass spectrometer (Micromass, Manchester, UK). To the collected samples were added 100 μL of distilled water, 25 μL of internal standard (500 ng/mL; 1-cyclohexyl-3-tetradecylurea, CTU), and 5000 μL of ethyl acetate. Then the samples were centrifuged at 6000 rpm for 5 min, and the ethyl acetate layer was dried under nitrogen. The residue was reconstituted in 25 μL of methanol, and aliquots (5 μL) were injected onto the LC-MS/MS system.

Analysis. Pharmacokinetics analysis was performed using SigmaPlot software system (SPSS science, Chicago, Ill.). A one-compartment model was used for blood concentration-time profiles for the oral gavage dosing and fits to the following equation (see, Gibson, G. G. and Skett, P.: INTRODUCTION TO DRUG METABOLISM, SECOND ED., Chapman and Hall, New York 1994, 199-210):

C=ae^{-bt}

The half-life (t_{1/2}) for the elimination phase was calculated by the following equation:

t_{1/2}=0.693/b

The area under the concentration (AUC) was calculated by the following equation:

AUC=a/b

### Where:

C=the total blood concentration at time t

a=the extrapolated zero intercept

b=the apparent first-order elimination rate constant

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A compound having a formula: 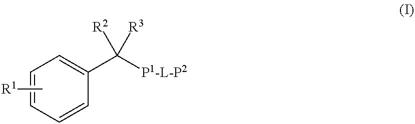
and the pharmaceutically acceptable salts, wherein
R¹ is a member selected from the group consisting of C₁-C₆ alkyl, C₁-C₆ hydroxyalkyl, C₁-C₆ haloalkyl, C₂-C₆ alkenyl, —C(O)—C₁-C₆ alkyl, C₁-C₆ alkyl-OSO₃H, C₃-C₆ cycloalkyl and an epoxy group optionally substituted with 1-2 groups each independently selected from the group consisting of H and C₁₋₆ alkyl;
each of R² and R³ are independently selected from the group consisting of C₁-C₆ alkyl and C₂-C₆ alkenyl, or R² and R³ are optionally combined to form a C₃-C₆ cycloalkyl;
P¹ is a primary pharmacophore of the formula —NH—C(O)—NH—;
L is a linker selected from the group consisting of C₁-C₁₂ alkylene, C₃-C₆ cycloalkylene, aryl-C₀-C₆ alkylene, C₃-C₆ cycloalkylene-O-aryl, C₀-C₆alkylenearyl-O-aryl and C₀-C₆-alkylene-C₃-C₆-heterocycloalkylene;
P² is a secondary pharmacophore selected from the group consisting of C₂-C₆ alkenyl, C₂-C₆ alkynyl, C₁-C₆ haloalkyl, aryl, heteroaryl, heterocyclyl, —O(CH₂CH₂O)_{q}—R⁴, —CN, —C(O)NHR⁴, —C(O)NHS(O)₂R⁴, —NHS(O)₂R⁴, —O—C₂-C₄alkyl-C(O)OR⁴, —C(O)R⁴, —C(O)OR⁴ and carboxylic acid analogs, wherein R⁴ is a member selected from the group consisting of hydrogen, C₁-C₆ alkyl, C₃-C₈ cycloalkyl, heterocyclyl, aryl and aryl-C₁-C₄ alkyl, or optionally P² is H; and
subscript q is from 1 to 6.

2. The compound of claim 1, having the formula: 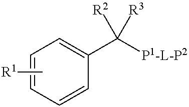
wherein
R¹ is a member selected from the group consisting of C₁-C₆ alkyl, C₂-C₆ alkenyl and C₃-C₆ cycloalkyl;
each of R² and R³ are independently selected from the group consisting of C₁-C₆ alkyl and C₂-C₆ alkenyl, or R² and R³ are optionally combined to form a C₃-C₆ cycloalkyl;
P¹ is a primary pharmacophore of the formula —NH—C(O)—NH—;
L is a linker selected from the group consisting of C₁-C₁₂ alkylene, C₃-C₆ cycloalkylene, aryl-C₀-C₆ alkylene, C₃-C₆ cycloalkylene-O-aryl, C₀-C₆alkylenearyl-O-aryl and C₀-C₆-alkylene-C₃-C₆-heterocycloalkylene;
P² is a secondary pharmacophore selected from the group consisting of C₂-C₆ alkenyl, C₂-C₆ alkynyl, C₁-C₆ haloalkyl, aryl, heteroaryl, heterocyclyl, —O(CH₂CH₂O)_{q}—R⁴, —CN, —C(O)NHR⁴, —C(O)NHS(O)₂R⁴, —NHS(O)₂R⁴, —O—C₂-C₄alkyl-C(O)OR⁴, —C(O)R⁴, —C(O)OR⁴ and carboxylic acid analogs, wherein R⁴ is a member selected from the group consisting of hydrogen, C₁-C₆ alkyl, C₃-C₈ cycloalkyl, heterocyclyl, aryl and aryl-C₁-C₄ alkyl, or optionally P² is H; and
subscript q is from 1 to 6.

3. The compound of claim 1, wherein:
R¹ is a member selected from the group consisting of C₁-C₆ alkyl and C₂-C₆ alkenyl;
and
each of R² and R³ are C ₁-C₆ alkyl.

4. The compound of claim 3, wherein R¹ is a member selected from the group consisting of isopropyl and isopropenyl.

5. The compound of claim 1, wherein L is a member selected from the group consisting of aryl-C₀-C₆ alkyl and C₃-C₆ cycloalkylene-O-aryl.

6. The compound of claim 5, wherein L is a member selected from the group consisting of phenyl-C₀-C₆ alkyl and cyclohexylene-O-phenyl.

7. The compound of claim 6, wherein L is phenyl-C₀-C₆ alkyl.

8. The compound of claim 6, wherein L is cyclohexylene-O-phenyl.

9. The compound of claim 1, wherein P² is a member selected from the group consisting of —CN and —C(O)OR⁴.

10. The compound of claim 9, wherein R⁴ is a member selected from the group consisting of hydrogen and C₁-C₄ alkyl.

11. The compound of claim 1, having Formula Ia:

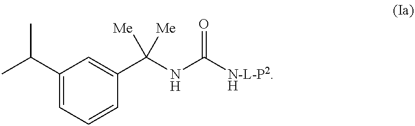

12. The compound of claim 11, selected from the group consisting of:

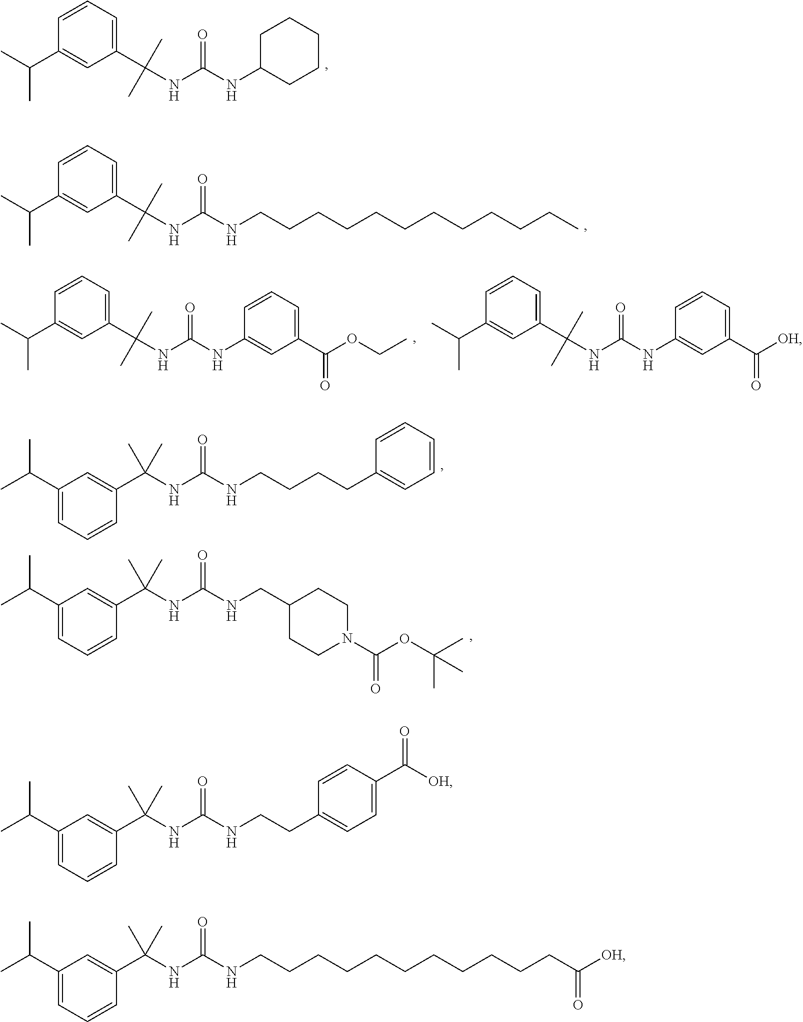

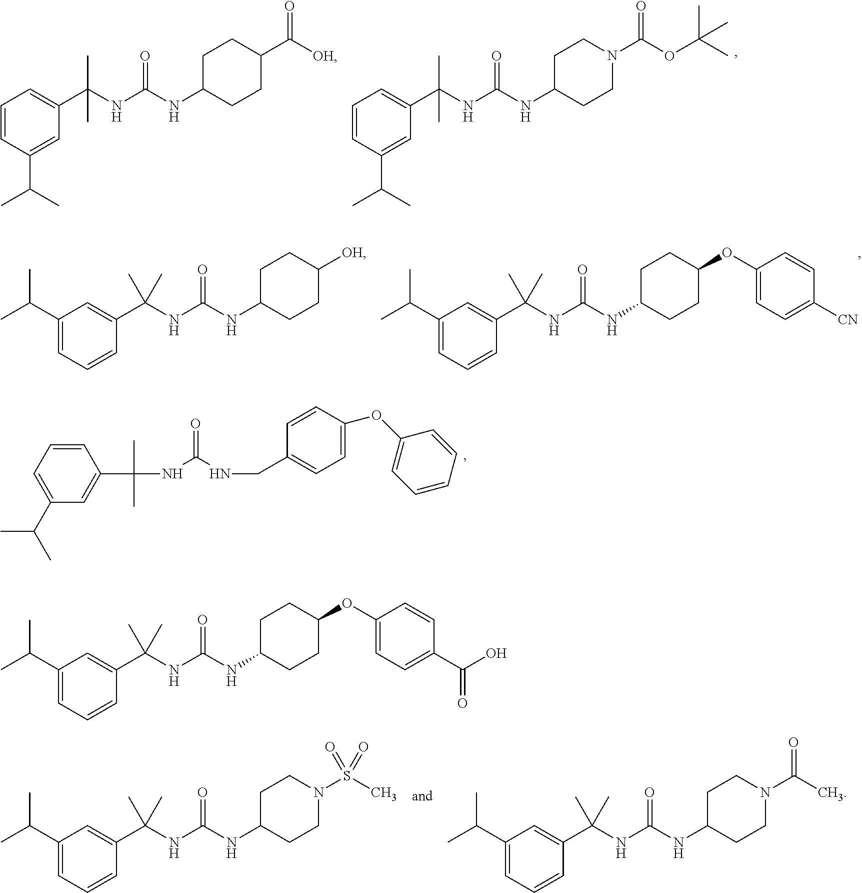

13. The compound of claim 12, selected from the group consisting of:

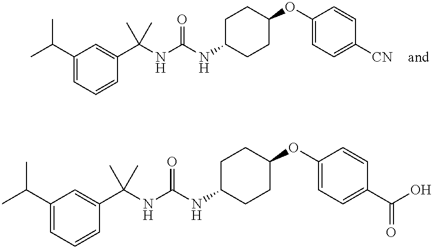

14. The compound of claim 1, having Formula Ib:

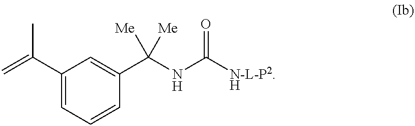

15. The compound of claim 14, selected from the group consisting of:

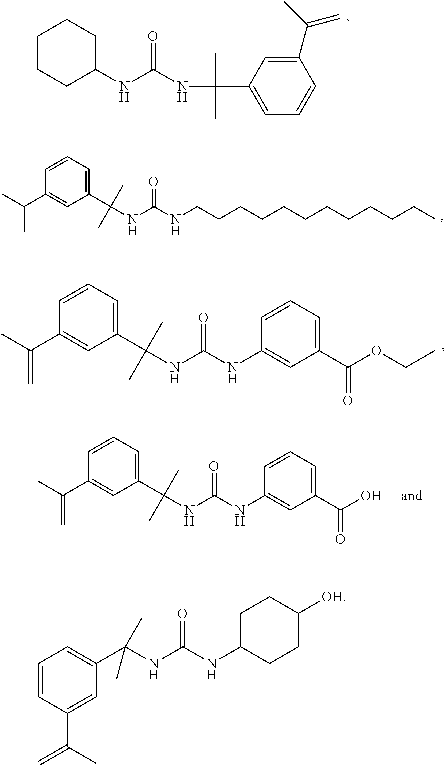

16. A pharmaceutical composition comprising a compound of claim 1 and a pharmaceutically acceptable excipient.

17. A method for inhibiting a soluble epoxide hydrolase, comprising contacting said soluble epoxide hydrolase with an inhibiting amount of a compound of claim 1.
